(19) United States
(12) Patent Application Publication
(10) Pub. No.: US 2003/0119062 A1
(43) Pub. Date: Jun. 26, 2003

(54) Detection of differential expression of protein using gel-free proteomics

(76) Inventor: Cynthia J. Brame, Charlottesville, VA (US)

Correspondence Address: ROPES & GRAY, ONE INTERNATIONAL PLACE, BOSTON, MA, 02110-2624, US

(21) Appl. No.: 10/211,945

(22) Filed: Aug. 2, 2002

Related U.S. Application Data
(60) Provisional application No. 60/309,903, filed on Aug. 3, 2001.

(51) Int. Cl. G01N 33/53; G06F 19/00; G01N 33/48; G01N 33/50

(52) U.S. Cl. 435/7.1; 702/19

(57) ABSTRACT

Methods and reagents for analyzing differential expression and/or abundance of distinct membrane-associated polypeptide samples, particularly integral membrane polypeptide samples are provided. Also provided are methods for screening pharmaceutical components that can affect expression or abundance of certain membrane-associated polypeptides; methods for identification of drug targets; and methods for diagnosis of certain disease states. Business methods for conducting a pharmaceutical business based on the result of using the above methods are also provided.

### REFERENCE TO RELATED APPLICATION

This application claims priority to U.S. Provisional Application No. 60/309,903, filed on Aug. 3, 2001, the entire content of which is incorporated by reference herein.

### BACKGROUND OF THE INVENTION

Proteomics holds great promise for determining polypeptides differentially expressed or regulated during different physiological or pathological conditions. Two-dimensional electrophoresis (2-DE), especially 2D-Polyacrylamide gel electrophoresis (2D-PAGE), is a highly useful resolving technique for separating and analyzing polypeptide samples based on their different molecular weights and isoelectric points. Due to its unsurpassed separation power, its use as the fundamental separation method for proteomics is warranted. The recent introduction of immobilized pH gradients (IPGs) for isoelectric focusing (IEF) is considered to be a milestone in the field of electrophoresis. Its benefits in 2-DE, including improved reproducibility, higher resolution, and increased capacity solidified the role of 2D-PAGE as a core polypeptide separation technology in proteomics.

However, current approaches relying on 2D-PAGE also have their limitations. While conventional 2D-PAGE are generally beneficial for analysis of soluble and mildly hydrophobic polypeptides, such techniques are particularly ill-suited for analyzing hydrophobic membrane polypeptides, such as cell surface receptors. The combination of poor solubility of these polypeptides and hydrophobic interactions between membrane polypeptides and the basic acrylamido derivatives of the IPG matrix frequently lead to poor resolution and lost sample spots, thus severely hampering the studying of an important family of polypeptides which are of strong interest in the pharmaceutical industry. Although significant efforts have been devoted to increase the solubility of membrane polypeptides, overall developments in 2-DE for membrane-associated polypeptides have been slow.

In addition, the large range of polypeptide expression levels limits the ability of the 2DE-MS approach to analyze polypeptides of medium to low abundance. Many polypeptides in a given proteome fall into that range, severely limiting the potential of this technique for proteome analysis. For instance in the case of yeast, fully one-half of all expressed yeast polypeptides are present at medium to low abundance, illustrating the importance of methods with large dynamic range.

Thus, there is a need to develop new methods for proteomic analysis of membrane-associated polypeptides.

### SUMMARY OF THE INVENTION

In general, the invention provides methods for identification of membrane-associated polypeptides, particularly integral membrane polypeptides, that exhibit altered abundance or post-translational modifications following certain treatments or transformations. The invention further provides methods for identification of compounds that can alter the abundance or post-translational modifications of a specific membrane polypeptide following treatment with those compounds.

In one aspect, the invention provides a method for identifying changes in membrane polypeptides, comprising: providing a test sample of membrane-associated polypeptides isolated from a test cell(s); by mass spectrometry using a quantitative mass analyzer, determining the levels of polypeptides in said test sample; comparing the level of one or more of the polypeptides from said test sample with levels of respective polypeptides from a reference sample; and identifying the sequences of polypeptides in the test sample which, relative to the reference sample, have altered abundance and/or altered levels of post-translational modification.

In one embodiment, the levels of polypeptides in said test sample is determined by Fourier-transform ion cyclotron resonance mass spectrometry (FTMS).

In another embodiment, the levels of polypeptides in said test sample is determined by Time-of-Flight mass spectrometry (TOF-MS).

In one embodiment, the membrane-associated polypeptides are cleaved to produce fragments including C-terminal arginine or lysine residues prior to analysis by mass spectrometry.

In one embodiment, the membrane-associated polypeptides are separated by chromatography prior to analysis by mass spectrometry. In a preferred embodiment, the chromatography is strong cation exchange chromatography.

In one embodiment, the mass spectrometry step includes ionizing the polypeptides of the test sample by electrospray ionization.

In one embodiment, the test sample is from a disease tissue and the reference sample is from a normal tissue.

In another embodiment, the polypeptides of the test sample are isolated based on post-translational modification. In a preferred embodiment, the polypeptides of the test sample are isolated based on phosphorylation.

In another aspect, the invention provides a method for identification of membrane-associated polypeptide targets of a compound, comprising: providing two test samples of membrane-associated polypeptides isolated from two test cells, wherein one test sample is an untreated reference sample and the other is a sample treated by said compound; by mass spectrometry using a quantitative mass analyzer, determining the levels of polypeptides in said test samples; comparing the level of one or more of the polypeptides from said treated test sample with levels of respective polypeptides from said reference sample; identifying the sequences of polypeptides in said treated sample which, relative to the reference sample, have altered abundance and/or altered levels of post-translational modification, thereby identifying the membrane-associated polypeptide targets of said compound.

In another aspect, the invention provides a method for identifying a compound which alters the abundance of a membrane-associated polypeptide in a sample, comprising: providing an reference sample and a plurality of test samples of membrane-associated polypeptides, each isolated from a test cell treated by a specific test compound; by mass spectrometry using a quantitative mass analyzer, determining the levels of said membrane-associated polypeptides in said test samples and said reference samples; comparing the level of one or more of said membrane-associated polypeptides from said test samples with levels of respective polypeptides from said reference sample; identifying the test sample which, relative to the reference sample, have altered abundance, thereby identifying the test compound responsible for the change.

In another aspect, the invention provides a method for identifying a compound which alters the levels of post-translational modification(s) of a membrane-associated polypeptide in a sample, comprising: providing an reference sample and a plurality of test samples of membrane-associated polypeptides, each isolated from a test cell treated by a specific test compound; by mass spectrometry using a quantitative mass analyzer, determining the levels of said membrane-associated polypeptides in said test samples and said reference samples; comparing the level of one or more of said membrane-associated polypeptides from said test samples with levels of respective polypeptides from said reference sample; identifying the test sample which, relative to the reference sample, have altered levels of post-translational modification(s), thereby identifying the test compound responsible for the change.

Yet another aspect of the present invention relates to a method of conducting a pharmaceutical business, comprising: by the above-described method, determining the identity of a target polypeptide isolated on the basis of the polypeptide (a) having a differential cellular localization of interest; (b) having a differential expression pattern of interest; (c) having a differential post-translational modification(s) of interest; or (d) having a differential abundance of interest; identifying compounds by their ability to alter the abundance or subcellular localization or post-translational modification(s) of the target polypeptide; conducting therapeutic profiling of compounds identified in step (ii), or further analogs thereof, for efficacy and toxicity in animals; and, formulating a pharmaceutical preparation including one or more compounds identified in step (iii) as having an acceptable therapeutic profile.

In a preferred embodiment, the business method further comprises an additional step of establishing a distribution system for distributing the pharmaceutical preparation for sale In yet another preferred embodiment, the business method further includes establishing a sales group for marketing the pharmaceutical preparation.

In another aspect, the invention provides a method of conducting a pharmaceutical business, comprising: by the above-described method, determining the identity of a target polypeptide isolated on the basis of the polypeptide: (a) having a differential cellular localization of interest, (b) having a differential expression pattern of interest, (c) having a differential post-translational modification(s) of interest, or (d) having a differential abundance of interest; optionally, conducting therapeutic profiling of the target gene for efficacy and toxicity in animals; and licensing, to a third party, the rights for further drug development of inhibitors or activators of the target gene.

### BRIEF DESCRIPTION OF THE FIGURES

FIG. 1. Schematic of differential analysis of membrane polypeptides.

FIG. 2. Identification of differentially expressed her2/neu peptides. Selected ion chromatograms of three her2/neu peptides from nanoHPLC/μESI/FTMS analysis of an SKBR3 ion exchange fraction are shown above; the level of these peptides were reduced at least 20-fold in the MCF-7 ion exchange fraction. CAD spectra of these peptides, shown at right, were obtained by targeted MS/MS on a quadrupole ion trap mass spectrometer, confirming identity of the peptides (3). The peptides are represented by SEQ ID NOs. 1-3.

### DETAILED DESCRIPTION OF THE INVENTION

### 1. Overview

In general, the invention provides methods for identification of membrane-associated polypeptides, particularly integral membrane polypeptides, and post-translationally modified polypeptides that exhibit altered abundance following certain treatments. The invention further provides methods for identification of compounds that can alter the abundance of a specific membrane polypeptide and post-translationally modified polypeptides following treatment with those compounds. In addition, the invention provides methods to compare a plurality of membrane-associated polypeptide samples for identification of polypeptides, the abundance or the level of post-translational modification of which are significantly altered among said samples. Particularly for comparison among samples obtained from disease and normal tissues, or treated and untreated tissues.

In one aspect, the invention provides a method for identifying changes in membrane polypeptides, comprising: providing a test sample of membrane-associated polypeptides isolated from a test cell(s); by mass spectrometry using a quantitative mass analyzer, determining the levels of polypeptides in said test sample; comparing the level of one or more of the polypeptides from said test sample with levels of respective polypeptides from a reference sample; and identifying the sequences of polypeptides in the test sample which, relative to the reference sample, have altered abundance and/or altered levels of post-translational modification(s).

In another aspect, the invention provides a method for identification of membrane-associated polypeptide targets of a compound, comprising: providing two test samples of membrane-associated polypeptides isolated from two test cells, wherein one test sample is an reference sample and the other is a sample treated by said compound; by mass spectrometry using a quantitative mass analyzer, determining the levels of polypeptides in said test samples; comparing the level of one or more of the polypeptides from said treated test sample with levels of respective polypeptides from said reference sample; identifying the sequences of polypeptides in said treated sample which, relative to the reference sample, have altered abundance and/or altered levels of post-translational modification(s), thereby identifying the membrane-associated polypeptide targets of said compound.

In another aspect, the invention provides a method for identifying a compound which alters the abundance of a membrane-associated polypeptide in a sample, comprising: providing an reference sample and a plurality of test samples of membrane-associated polypeptides, each isolated from a test cell treated by a specific test compound; by mass spectrometry using a quantitative mass analyzer, determining the levels of said membrane-associated polypeptides in said test samples and said reference samples; comparing the level of one or more of said membrane-associated polypeptides from said test samples with levels of respective polypeptides from said reference sample; identifying the test sample which, relative to the reference sample, have altered abundance, thereby identifying the test compound responsible for the change.

In another aspect, the invention provides a method for identifying a compound which alters the levels of post-translational modification of a membrane-associated polypeptide in a sample, comprising: providing an reference sample and a plurality of test samples of membrane-associated polypeptides, each isolated from a test cell treated by a specific test compound; by mass spectrometry using a quantitative mass analyzer, determining the levels of said membrane-associated polypeptides in said test samples and said reference samples; comparing the level of one or more of said membrane-associated polypeptides from said test samples with levels of respective polypeptides from said reference sample; identifying the test sample which, relative to the reference sample, have altered levels of post-translational modification, thereby identifying the test compound responsible for the change.

The membrane-associated polypeptides and post-translationally modified polypeptides can be isolated and/or fractionated using a variety of methods. The isolated polypeptide of interest, either modified or unmodified, can then be digested and separated before used for differential analysis. In a preferred embodiment of the invention, digested polypeptide samples are separated by strong cation exchange chromatography into multiple fractions (usually into about 10 fractions) so that the complexity of each fraction is amenable to subsequent FTMS differential analysis. In another preferred embodiment, IMAC is used to isolate a subset of all polypeptides, for example, phosphopeptides. These fractionated samples are then subjected to analysis by nanoHPLC (high performance liquid chromatography), and are directly introduced into a quantitative mass analyzer following μESI (micro-electrospray ionization). A Fourier-transform ion cyclotron resonance mass spectrometer (FTMS) can be used to obtain high resolution mass spectra with a large dynamic range, which is ideal for subsequent generation of a list of all polypeptide fragments of interest. These lists of polypeptides from different samples can be compared and any peptide fragments exhibiting substantial alteration in abundance can be further isolated and their sequences identified by tandem mass spectrometry using an LCQ ion trap mass spectrometer or equivalent instruments.

These methods holds great potential for a variety of useful purposes. For example, they can be used to identify cell-surface (membrane-associated) disease markers, thus providing useful diagnosis/prognosis tools. They can be used to screen for antagonists or agonists of certain membrane-associated polypeptides whose abundance changes following treatments by those antagonists/agonists. They can be used to identify polypeptide targets of certain compounds, which are known to have certain defined biological activity in cells but the polypeptide targets of which remain elusive. They can also be used to track changes in phosphorylation and other post-translational modifications of certain polypeptides following certain treatments, thereby providing useful clues as to which signal transduction pathways are activated/inactivated following those treatments. These kind of information will help to rapidly identify further markers for diagnosis and drug targets for treatment of certain disease.

Yet another aspect of the present invention relates to a method of conducting a pharmaceutical business, comprising: by the above-described method, determining the identity of a target polypeptide isolated on the basis of the polypeptide being (a) having a differential cellular localization of interest; (b) having a differential expression pattern of interest; (c) having a differential post-translational modification(s) of interest; or (d) having a differential abundance of interest; identifying compounds by their ability to alter the abundance or subcellular localization or post-translational modification of the target polypeptide; conducting therapeutic profiling of compounds identified in step (ii), or further analogs thereof, for efficacy and toxicity in animals; and, formulating a pharmaceutical preparation including one or more compounds identified in step (iii) as having an acceptable therapeutic profile.

In a preferred embodiment, the business method further comprises an additional step of establishing a distribution system for distributing the pharmaceutical preparation for sale In yet another preferred embodiment, the business method further including establishing a sales group for marketing the pharmaceutical preparation.

In another aspect, the invention provides a method of conducting a pharmaceutical business, comprising: by the above-described method, determining the identity of a target polypeptide isolated on the basis of the polypeptide: (a) having a differential cellular localization of interest, (b) having a differential expression pattern of interest, (c) having a differential post-translational modification of interest, or (d) having a differential abundance of interest; optionally, conducting therapeutic profiling of the target gene for efficacy and toxicity in animals; and licensing, to a third party, the rights for further drug development of inhibitors or activators of the target gene.

### 2. Definitions

“Altered” or “significantly altered” is meant that there is a quantitative difference of at least two-fold, preferably 5-fold, more preferably 10-fold, and most preferably 50-fold. The altered abundance can either be increased or decreased as compared to wild-type or control/reference samples.

“Abundance” as used herein is meant “level” or steady state level or amount.

“Membrane” or “membrane-associated” as used herein is meant membrane-associated, either constitutively or induced. A membrane polypeptide can be an integral membrane polypeptide; a polypeptide associated with membrane indirectly via a non-polypeptide moiety, such as GPI-linker, prenylation, myristoylation, or palmitoylation; or a polypeptide associated with membrane indirectly via binding to another membrane-associated polypeptide. The membrane association can be constitutive, or be induced by certain signaling event-induced changes, such as polypeptide phosphorylation/dephosphorylation, activation by associating with an active form of a molecule rather than a previous inactive form of a molecule (i.e. GTP-bound vs. GDP-bound), conformation change, activation by partial proteolysis, and other post-translational modifications.

“Homology” or “identity” or “similarity” refers to sequence similarity between two peptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the sequences of the present invention.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both polypeptide and DNA databases.

Databases with individual sequences are described in Methods in Enzymology, ed. Doolittle, supra. Databases include Genbank, EMBL, and DNA Database of Japan (DDBJ). In comparing a new nucleic acid with known sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489.

The terms “protein”, “polypeptide” and “peptide” are used interchangeably herein when referring to a natural or recombinant gene product of fragment thereof.

The term “recombinant protein” refers to a polypeptide of the present invention which is produced by recombinant DNA techniques, wherein generally, DNA encoding a polypeptide is inserted into a suitable expression vector which is in turn used to transform a host cell to produce the heterologous polypeptide. Moreover, the phrase “derived from”, with respect to a recombinant gene, is meant to include within the meaning of “recombinant protein” those polypeptides having an amino acid sequence of a native polypeptide, or an amino acid sequence similar thereto which is generated by mutations including substitutions and deletions (including truncation) of a naturally occurring form of the polypeptide.

“Small molecule” as used herein, is meant to refer to a composition which has a molecular weight of less than about 5 kDa and most preferably less than about 4 kDa. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

Genetic techniques, which allow for the expression of transgenes can be regulated via site-specific genetic manipulation in vivo, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject target gene polypeptides. For example, excision of a target sequence which interferes with the expression of a recombinant target gene, such as one which encodes an antagonistic homolog or an antisense transcript, can be designed to activate expression of that gene. This interference with expression of the polypeptide can result from a variety of mechanisms, such as spatial separation of the target gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allows for promoter driven transcriptional activation.

“Protein target” is meant direct or indirect target of a given compound. The compound may directly bind to the polypeptide target, or indirectly cause alterations in abundance of the polypeptide target in cell following treatment by the compound. There could be more than one intermediate components in the chain of reaction between the stimulation by a compound and the alteration in the abundance of the polypeptide target.

“Phospho-protein” is meant a polypeptide that can be potentially phosphorylated on at least one residue, which can be either tyrosine or serine or threonine or any combination of the three. Phosphorylation can occur constitutively or be induced.

“Post-translational modification” is meant any changes/modifications that can be made to the native polypeptide sequence after its initial translation. It includes, but are not limited to, phosphorylation/dephosphorylation, prenylation, myristoylation, palmitoylation, limited digestion, irreversible conformation change, methylation, acetylation, modification to amino acid side chains or the amino terminus, and changes in oxidation, disulfide-bond formation, etc.

### 3. Isolation of Polypeptides

Historically, polypeptide purification schemes have been predicated on differences in the molecular properties of size, charge and solubility between the polypeptide to be purified and undesired polypeptide contaminants. Protocols based on these parameters include size exclusion chromatography, ion exchange chromatography, differential precipitation and the like.

Size exclusion chromatography, otherwise known as gel filtration or gel permeation chromatography, relies on the penetration of macromolecules in a mobile phase into the pores of stationary phase particles. Differential penetration is a function of the hydrodynamic volume of the particles. Accordingly, under ideal conditions the larger molecules are excluded from the interior of the particles while the smaller molecules are accessible to this volume and the order of elution can be predicted by the size of the polypeptide because a linear relationship exists between elution volume and the log of the molecular weight. Size exclusion chromatographic supports based on cross-linked dextrans e.g. SEPHADEX.RTM., spherical agarose beads e.g. SEPHAROSE.RTM. (both commercially available from Pharmacia AB. Uppsala, Sweden), based on cross-linked polyacrylamides e.g. BIO-GEL.RTM. (commercially available from BioRad Laboratories, Richmond, Calif.) or based on ethylene glycol-methacrylate copolymer e.g. TOYOPEARL HW65S (commercially available from ToyoSoda Co., Tokyo, Japan) are useful in the practice of this invention.

Precipitation methods are predicated on the fact that in crude mixtures of polypeptides the solubilities of individual polypeptides are likely to vary widely. Although the solubility of a polypeptide in an aqueous medium depends on a variety of factors, for purposes of this discussion it can be said generally that a polypeptide will be soluble if its interaction with the solvent is stronger than its interaction with polypeptide molecules of the same or similar kind. Without wishing to be bound by any particular mechanistic theory describing precipitation phenomena, it is nonetheless believed that the interaction between a polypeptide and water molecules occur by hydrogen bonding with several types of charged groups, and electrostatically as dipoles with uncharged groups, and that precipitants such as salts of monovalent cations (e.g., ammonium sulfate) compete with polypeptides for water molecules, thus at high salt concentrations, the polypeptides become “dehydrated” reducing their interaction with the aqueous environment and increasing the aggregation with like or similar polypeptides resulting in precipitation from the medium.

Ion exchange chromatography involves the interaction of charged functional groups in the sample with ionic functional groups of opposite charge on an adsorbent surface. Two general types of interaction are known. Anionic exchange chromatography mediated by negatively charged amino acid side chains (e.g. aspartic acid and glutamic acid) interacting with positively charged surfaces and cationic exchange chromatography mediated by positively charged amino acid residues (e.g. lysine and arginine) interacting with negatively charged surfaces.

More recently affinity chromatography and hydrophobic interaction chromatography techniques have been developed to supplement the more traditional size exclusion and ion exchange chromatographic protocols. Affinity chromatography relies on the interaction of the polypeptide with an immobilized ligand. The ligand can be specific for the particular polypeptide of interest in which case the ligand is a substrate, substrate analog, inhibitor or antibody. Alternatively, the ligand may be able to react with a number of polypeptides. Such general ligands as adenosine monophosphate, adenosine diphosphate, nicotine adenine dinucleotide or certain dyes may be employed to recover a particular class of polypeptides. One of the least biospecific of the affinity chromatographic approaches is immobilized metal affinity chromatography (IMAC), also referred to as metal chelate chromatography. IMAC introduced by Porath et al.(Nature 258:598-99(1975) involves chelating a metal to a solid support and then forming a complex with electron donor amino acid residues on the surface of a polypeptide to be separated.

Hydrophobic interaction chromatography was first developed following the observation that polypeptides could be retained on affinity gels which comprised hydrocarbon spacer arms but lacked the affinity ligand. Although in this field the term hydrophobic chromatography is sometimes used, the term hydrophobic interaction chromatography (HIC) is preferred because it is the interaction between the solute and the gel that is hydrophobic not the chromatographic procedure. Hydrophobic interactions are strongest at high ionic strength, therefore, this form of separation is conveniently performed following salt precipitations or ion exchange procedures. Elution from HIC supports can be effected by alterations in solvent, pH, ionic strength, or by the addition of chaotropic agents or organic modifiers, such as ethylene glycol. A description of the general principles of hydrophobic interaction chromatography can be found in U.S. Pat. No. 3,917,527 and in U.S. Pat. No. 4,000,098. The application of HIC to the purification of specific polypeptides is exemplified by reference to the following disclosures: human growth hormone (U.S. Pat. No. 4,332,717), toxin conjugates (U.S. Pat. No. 4,771,128), antihemolytic factor (U.S. Pat. No. 4,743,680), tumor necrosis factor (U.S. Pat. No. 4,894,439), interleukin-2 (U.S. Pat. No. 4,908,434), human lymphotoxin (U.S. Pat. No. 4,920,196) and lysozyme species (Fausnaugh, J. L. and F. E. Regnier, J. Chromatog. 359:131-146 (1986)).

The principles of IMAC are generally appreciated. It is believed that adsorption is predicated on the formation of a metal coordination complex between a metal ion, immobilized by chelation on the adsorbent matrix, and accessible electron donor amino acids on the surface of the polypeptide to be bound. The metal-ion microenvironment including, but not limited to, the matrix, the spacer arm, if any, the chelating ligand, the metal ion, the properties of the surrounding liquid medium and the dissolved solute species can be manipulated by the skilled artisan to affect the desired fractionation.

Not wishing to be bound by any particular theory as to mechanism, it is further believed that the more important amino acid residues in terms of binding are histidine, tryptophan and probably cysteine. Since one or more of these residues are generally found in polypeptides, one might expect all polypeptides to bind to IMAC columns. However, the residues not only need to be present but also accessible (e.g., oriented on the surface of the polypeptide) for effective binding to occur. Other residues, for example poly-histidine tails added to the amino terminus or carboxyl terminus of polypeptides, can be engineered into the recombinant expression systems by following the protocols described in U.S. Pat. No. 4,569,794.

The nature of the metal and the way it is coordinated on the column can also influence the strength and selectivity of the binding reaction. Matrices of silica gel, agarose and synthetic organic molecules such as polyvinyl-methacrylate co-polymers can be employed. The matrices preferably contain substituents to promote chelation. Substituents such as iminodiacetic acid (IDA) or its tris (carboxymethyl) ethylene diamine (TED) can be used. IDA is preferred. A particularly useful IMAC material is a polyvinyl methacrylate co-polymer substituted with IDA available commercially, e.g., as TOYOPEARL AF-CHELATE 650M (ToyoSoda Co.; Tokyo. The metals are preferably divalent members of the first transition series through to zinc, although Co⁺⁺, Ni⁺⁺, Cd⁺⁺ and Fe⁺⁺⁺ can be used. An important selection parameter is, of course, the affinity of the polypeptide to be purified for the metal. Of the four coordination positions around these metal ions, at least one is occupied by a water molecule which is readily replaced by a stronger electron donor such as a histidine residue at slightly alkaline pH.

In practice the IMAC column is “charged” with metal by pulsing with a concentrated metal salt solution followed by water or buffer. The column often acquires the color of the metal ion (except for zinc). Often the amount of metal is chosen so that approximately half of the column is charged. This allows for slow leakage of the metal ion into the non-charged area without appearing in the eluate. A pre-wash with intended elution buffers is usually carried out. Sample buffers may contain salt up to 1M or greater to minimize nonspecific ion-exchange effects. Adsorption of polypeptides is maximal at higher pHs. Elution is normally either by lowering of pH to protonate the donor groups on the adsorbed polypeptide, or by the use of stronger complexing agent such as imidazole, or glycine buffers at pH 9. In these latter cases the metal may also be displaced from the column. Linear gradient elution procedures can also be beneficially employed.

As mentioned above, IMAC is particularly useful when used in combination with other polypeptide fractionation techniques. That is to say it is preferred to apply IMAC to material that has been partially fractionated by other protein fractionation procedures. A particularly useful combination chromatographic protocol is disclosed in U.S. Pat. No. 5,252,216 granted Oct. 12, 1993, the contents of which are incorporated herein by reference. It has been found to be useful, for example, to subject a sample of conditioned cell culture medium to partial purification prior to the application of IMAC. By the term “conditioned cell culture medium” is meant a cell culture medium which has supported cell growth and/or cell maintenance and contains secreted product. A concentrated sample of such medium is subjected to one or more polypeptide purification steps prior to the application of a IMAC step. The sample may be subjected to ion exchange chromatography as a first step. As mentioned above various anionic or cationic substituents may be attached to matrices in order to form anionic or cationic supports for chromatography. Anionic exchange substituents include diethylaminoethyl (DEAE), quaternary aminoethyl (QAE) and quaternary amine (Q) groups. Cationic exchange substituents include carboxymethyl (CM), sulfoethyl (SE), sulfopropyl (SP), phosphate (P) and sulfonate (S). Cellulosic ion exchange resins such as DE23, DE32, DE52, CM-23, CM-32 and CM-52 are available from Whatman Ltd. Maidstone, Kent, U.K. SEPHADEX.RTM.-based and cross-linked ion exchangers are also known. For example, DEAE-, QAE-, CM-, and SP-dextran supports under the tradename SEPHADEX.RTM. and DEAE-, Q-, CM- and S-agarose supports under the tradename SEPHAROSE.RTM. are all available from Pharmacia AB. Further both DEAE and CM derivitized ethylene glycol-methacrylate copolymer such as TOYOPEARL DEAE-650S and TOYOPEARL CM-650S are available from Toso Haas Co., Philadelphia, Pa. Because elution from ionic supports sometimes involves addition of salt and IMAC may be enhanced under increased salt concentrations. The introduction of a IMAC step following an ionic exchange chromatographic step or other salt mediated purification step may be employed. Additional purification protocols may be added including but not necessarily limited to HIC, further ionic exchange chromatography, size exclusion chromatography, viral inactivation, concentration and freeze drying.

Hydrophobic molecules in an aqueous solvent will self-associate. This association is due to hydrophobic interactions. It is now appreciated that macromolecules such as polypeptides have on their surface extensive hydrophobic patches in addition to the expected hydrophilic groups. HIC is predicated, in part, on the interaction of these patches with hydrophobic ligands attached to chromatographic supports. A hydrophobic ligand coupled to a matrix is variously referred to herein as an HIC support, HIC gel or HIC column. It is further appreciated that the strength of the interaction between the polypeptide and the HIC support is not only a function of the proportion of non-polar to polar surfaces on the polypeptide but by the distribution of the non-polar surfaces as well.

A number of matrices may be employed in the preparation of HIC columns, the most extensively used is agarose. Silica and organic polymer resins may be used. Useful hydrophobic ligands include but are not limited to alkyl groups having from about 2 to about 10 carbon atoms, such as a butyl, propyl, or octyl; or aryl groups such as phenyl. Conventional HIC products for gels and columns may be obtained commercially from suppliers such as Pharmacia LKB AB, Uppsala, Sweden under the product names butyl-SEPHAROSE.RTM., phenyl-SEPHAROSE.RTM. CL-4B, octyl-SEPHAROSE.RTM. FF and phenyl-SEPHAROSE.RTM. FF; Tosoh Corporation, Tokyo, Japan under the product names TOYOPEARL Butyl 650, Ether-650, or Phenyl-650 (FRACTOGEL TSK Butyl-650) or TSK-GEL phenyl-5PW; Miles-Yeda, Rehovot, Israel under the product name ALKYL-AGAROSE, wherein the alkyl group contains from 2-10 carbon atoms, and J. T. Baker, Phillipsburg, N. J. under the product name BAKERBOND WP-HI-propyl.

Ligand density is an important parameter in that it influences not only the strength of the interaction but the capacity of the column as well. The ligand density of the commercially available phenyl or octyl phenyl gels is on the order of 40 μM/ml gel bed. Gel capacity is a function of the particular polypeptide in question as well pH, temperature and salt concentration but generally can be expected to fall in the range of 3-20 mg/ml of gel.

The choice of a particular gel can be determined by the skilled artisan. In general the strength of the interaction of the polypeptide and the HIC ligand increases with the chain length of the of the alkyl ligands but ligands having from about 4 to about 8 carbon atoms are suitable for most separations. A phenyl group has about the same hydrophobicity as a pentyl group, although the selectivity can be quite different owing to the possibility of pi-pi interaction with aromatic groups on the polypeptide.

Adsorption of the polypeptides to a HIC column is favored by high salt concentrations, but the actual concentrations can vary over a wide range depending on the nature of the polypeptide and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba⁺⁺<Ca⁺⁺<Mg⁺⁺<Li⁺<Cs⁺<Na⁺<K⁺<Rb⁺<NH₄⁺. While anions may be ranked in terms of increasing chaotropic effect as PO₄⁻⁻⁻<SO₄⁻⁻<CH₃COO⁻<Cl⁻<Br⁻<NO₃⁻<CIO₄⁻<I⁻<SCN⁻.

Accordingly, salts may be formulated that influence the strength of the interaction as given by the following relationship:

Na₂SO₄>NaCl>(NH₄)₂SO₄>NH₄Cl>NaBr>NaSCN

In general, salt concentrations of between about 0.75 and about 2M ammonium sulfate or between about 1 and 4M NaCl are useful.

The influence of temperature on HIC separations is not simple, although generally a decrease in temperature decreases the interaction. However, any benefit that would accrue by increasing the temperature must also be weighed against adverse effects such an increase may have on the activity of the polypeptide.

Elution, whether stepwise or in the form of a gradient, can be accomplished in a variety of ways: (a) by changing the salt concentration, (b) by changing the polarity of the solvent or (c) by adding detergents. By decreasing salt concentration adsorbed polypeptides are eluted in order of increasing hydrophobicity. Changes in polarity may be affected by additions of solvents such as ethylene glycol or (iso)propanol thereby decreasing the strength of the hydrophobic interactions. Detergents function as displacers of polypeptides and have been used primarily in connection with the purification of membrane polypeptides.

When the eluate resulting from HIC is subjected to further ion exchange chromatography, both anionic and cationic procedures may be employed.

As mentioned above, gel filtration chromatography affects separation based on the size of molecules. It is in effect a form of molecular sieving. It is desirable that no interaction between the matrix and solute occur, therefore, totally inert matrix materials are preferred. It is also desirable that the matrix be rigid and highly porous. For large scale processes rigidity is most important as that parameter establishes the overall flow rate. Traditional materials such as crosslinked dextran or polyacrylamide matrices, commercially available as, e.g., SEPHADEX.RTM. and BIOGEL.RTM., respectively, were sufficiently inert and available in a range of pore sizes, however these gels were relatively soft and not particularly well suited for large scale purification. More recently, gels of increased rigidity have been developed (e.g. SEPHACRYL.RTM., ULTROGEL.RTM., FRACTOGEL.RTM. and SUPEROSE.RTM.). All of these materials are available in particle sizes which are smaller than those available in traditional supports so that resolution is retained even at higher flow rates. Ethylene glycol-methacrylate copolymer matrices, e.g., such as the TOYOPEARL HW series matrices (Toso Haas) are preferred.

Phosphoproteins can be isolated using IMAC as described above. However, they can also be isolated by other means. Specifically, phosphoproteins with phosphorylated tyrosine residues can be isolated with phospho-tyrosine specific antibodies. Likewise, phospho-serine/threonine specific antibodies can be used to isolate phosphoproteins with phosphorylated serine/threonine residues. Many of these antibodies are available as affinity purified forms, either as monoclonal antibodies or antisera or mouse ascites fluid. For example, phospho-Tyrosine monoclonal antibody (P-Tyr-102) is a high-affinity IgGI phospho-tyrosine antibody clone that is produced and characterized by Cell Signaling Technology (Beverly, Mass.). As determined by ELISA, PTyr-102 (Cat. No. 9416) binds to a larger number of phospho-tyrosine containing peptides in a manner largely independent of the surrounding amino acid sequences, and also interacts with a broader range of phospho-tyrosine containing polypeptides as indicated by 2D-gel Western analysis. P-Tyr-102 is highly specific for phospho-Tyr in peptides/proteins, shows no cross-reactivity with the corresponding nonphosphorylated peptides and does not react with peptides containing phospho-Ser or phospho-Thr instead of phospho-Tyr. It is expected that P-Tyr-102 will react with peptides/proteins containing phospho-Tyr from all species.

Phospho-threonine antibodies are also available. For example, Cell Signaling Technology also offer an affinity-purified rabbit polyclonal phospho-threonine antibody (P-Thr-Polyclonal, Cat. No. 9381) which binds threonine-phosphorylated sites in a manner largely independent of the surrounding amino acid sequence. It recognizes a wide range of threonine-phosphorylated peptides in ELISA and a large number of threonine-phosphorylated polypeptides in 2D analysis. It is specific for peptides/proteins containing phospho-Thr and shows no cross-reactivity with corresponding nonphosphorylated sequences. Phospho-Threonine Antibody (P-Thr-Polyclonal) does not cross-react with sequences containing either phospho-Tyrosine or phospho-Serine. It is expected that this antibody will react with threonine-phosphorylated peptides/proteins regardless of species of origin. Upstate Biotechnology (Lake Placid, N.Y.) also provides an anti-phosphoserine/threonine antibody with broad immunoreactivity for polypeptides containing phosphorylated serine and phosphorylated threonine residues.

Many other similar products are also available on the market. These antibodies can be readily coupled to supporting matrix materials to generate affinity columns according to standard molecular biology protocols (see Using Antibodies: A Laboratory Manual: Portable Protocol NO. I, Harlow and Lane, Cold Spring Harbor Laboratory Press: 1998; also see Antibodies: A Laboratory Manual, edited by Harlow and Lane, Cold Spring Harbor Laboratory Press: 1988).

A similar approach can be applied towards the isolation of any specific polypeptide, against which specific antibodies are available.

Isolation of membrane-associated polypeptides can be carried out using appropriate methods as described above (for example, hydrophobic interaction chromatography). Alternatively, it can be performed with other standard molecular biology protocols. See, for example, Molecular Cloning A Laboratory Manual, 2nd Ed., ed. by Sambrook, Fritsch and Maniatis (Cold Spring Harbor Laboratory Press: 1989); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987).

For example, cells can be lysed in appropriate buffers and the membrane portions can be isolated by centrifugation. Depending on particular cases, cells preferably can be lysed in hypotonic buffer by homogenization. Cell debris and nuclei can then be removed by low speed centrifugation, followed by high speed centrifugation (such as under centrifugation conditions of 100,000×g or more) to pellet membrane portions. Membrane polypeptides can then be extracted by organic solvents such as chloroform and methanol.

Alternatively, membrane polypeptides can be isolated by extraction of membrane portions with extraction buffer containing detergents. Depending on specific occasions, the detergent used can be SDS or other ionic or non-ionic detergents. Different choices of detergent or extraction buffer in general may facilitate global non-biased extraction of membrane polypeptides or isolation of specific membrane polypeptides of interest. The reduced complexity of polypeptide mixtures resulting from the use of specific extraction protocols may be beneficial for the following digestion, separation, and analysis procedures.

A most preferred method of isolating hydrophobic membrane proteins is strong cation exchange (SCX) chromatography. Strong cation exchange (SCX) chromatography is particularly suited for isolating/purifying hydrophobic proteins, such as membrane proteins. Many SCX chromatographic columns are commercially available. For illustration purpose only, details regarding one type of SCX column, the PolySulfoethyl Aspartamide Strong Cation Exchange Columns manufactured by The Nest Group, Inc. (45 Valley Road, Southborough, Mass.), are described below. It is to be understood that the recommendations below are by no means limiting in any respect. Many other commercial SCX columns are also available, and should be used according to the recommendation of respective manufacturers.

According to the manufacturer, aspartamide cation exchange chemistries are some of the best materials available for the HPLC separation of peptides. These are wide-pore (300 Å) silica packings with a bonded coating of hydrophilic, sulfoethyl anionic polymer. With the PolySULFOETHYL Aspartamide SCX column, mobile phase modifiers can be used to help improve peptide solubility or to mediate the interaction between peptide and stationary phase. By varying the pH, ionic strength or organic solvent concentration in the mobile phase, chromatographic selectivity can be significantly enhanced. For more strongly hydrophobic peptides, a non-ionic surfactant (at a concentration below its CMC) and/or acetonitrile or n-propanol as mobile phase modifiers, can substantially improve resolution and recovery over conventional reverse phase methods. Additional selectivity can be obtained by simply changing the slope of the KCl or (NH₄)₂SO₄ gradient.

Using this column at pH 3 is better for retention of neutral to slightly acidic peptides. Use of a higher pH may be considered for basic hydrophobic peptides. The addition of MeCN or propanol to the A&B solvents (see below) changes the mechanism of separation and results in a separation based not only on positive charge, but also on hydrophobicity.

These columns are quite useful for neuropeptides, growth factors, CNBr peptide fragments, and synthetic peptides as a complement to RPC (Reverse Phase Chromatography), or to remove organic reagents from peptide samples which would cause smearing on a RPC column.

The operating conditions for these applications for an analytical column are:

Buffer A: 5 mM K-PO₄+25% MeCN;

Buffer B: 5 mM K-PO₄+25% MeCN+300-500 mM KCl;

Linear gradient, 30 min at 1 ml/min.

The peptides are retained on the column by the positive charge of at least the terminus amino and elute by total charge, charge distribution and hydrophobicity. If the peptide does not stick to the column, prepare the peptide in a small amount of buffer, or decrease the concentration of organic in the A&B solvents to 5 or 10%. Organic solvent concentration is empirically determined and n-propanol can be substituted for MeCN for more hydrophobic species.

Since the total binding capacity of these columns is on the order of 100 mg/gm of packing (for nonresolved materials) there will be a considerable Donan effect present. It will be necessary to have the sample in 5-15 mM of salt or buffer to prevent exclusion from the column. Additionally, the gradient at the outlet of the column will be much more concave than that observed on the chart paper. It is recommended that an upper load limit of 1 milligram for an analytical column. For a guard column used as a methods development column, a load limit of one-tenth of a milligram is recommended.

Flow rates of 0.7 to 1.0 m/min with a 30 minutes gradient should be used for the analytical column. If using the 4.6×20 mm guard column as a methods development column, gradient times should be shortened to 8-10 min at the same flow rate since the void volume is only 0.3 ml. The semiprep columns, 9.4 mm ID, require flow rates and equilibration volumes 4× that of the analytical columns.

Typically, for the first run, equilibrate the analytical column in the high salt (or final pH) solution (at least 25 ml, or for a guard column used as a methods development column use 8 ml, or on the semiprep column use 100 ml), and inject the sample under these isocratic conditions to observe the elution profile. The protein should elute at the void volume. Then equilibrate the column in low salt (or low pH if doing a pH gradient) conditions and run the gradient to the final conditions. Comparison of the chromatograms will assure that the proteins will elute in a predictable fashion. To decrease elution times increase the salt concentration (in a convex or step manner), increase the pH, or shorten the equilibration times between gradient runs. Exposure to a pH above 7 should be avoided since this will affect the silica support and will shorten column life, as will temperatures above 45° C. For buffer gradients, phosphate or bis-tris are good buffers to use since they allow monitoring in the low UV range. For salt gradients, acetate salts are frequently used. However, it may be necessary to use sulfate or chloride if the buffering capacity of acetate is undesirable or if the absorbance is to be monitored below 235 nm. When chloride has been used for salt gradient elution, flush the column with at least 30 ml of deionized water at the end of the day to prevent corrosion. If a denaturant such as 4M urea is used in the mobile phase to increase the accessibility of the ionizable groups, be sure to have a silica saturator column in line in front of the injector, to minimize attack of the silica on the ion exchange column.

New columns should be condition before use, preferably according to the following protocol. Specifically, columns are filled with methanol when shipped so the (analytical) column should be flushed with at least 40 ml water before elution with salt solution to prevent precipitation. The hydrophilic coating imbibes a layer of water. The resultant swelling of the coating leads to a slight and irreversible increase in the column back pressure. Some additional swelling occurs with extended use of the column. Since the swelling increases the surface area of the coating, the capacity of the column for proteins increases as well. Thus, retention times may increase by up to 10%. This process should be hastened by eluting the column with a strong buffer for at least one hour prior to its initial use. A convenient solution to use is 0.2 M monosodium phosphate+0.3 M sodium acetate.

The conditioning process is reversed by exposing the column to pure organic solvents. Accordingly, to minimize the time to start the column after a 1-2 day storage, the column should be flushed with at least 40 ml of deionized water (not methanol), and the ends should be plugged. For extended storage it is recommended that a 100% methanol storage be used to prevent bacterial growth and contamination. Exercise care when using organic solvents to prevent precipitation of salts.

It is recommended that a new column be conditioned with two injections of an inexpensive protein (e.g. BSA) before it is used to analyze very dilute or expensive samples since new HPLC columns sometimes absorb small quantities of proteins in a nonspecific manner. The scintered metal frits have been implicated in this process. Fortunately these sites are quickly saturated. Mobile phases should be filtered before use, as should samples. Failure to do so may cause the inlet frit to plug. A guard column, P410-2SEA, will prevent damage to the analytical or preparative columns. Use of 0.1% TFA or high concentrations of formic acid in the mobile phase is not recommended.

For use in normal phase and HILIC polarity, the following should be taken into consideration. By adding even more organic solvent to the mobile phase, these columns offer enough flexibility so that they may be used in a normal or Hydrophilic Interaction (HLIC) mode. Here, more polar peptides having little or no retention under conventional reverse-phase or even ion-exchange conditions are retained, and very hydrophobic peptides may have enhanced solubility and thus chromatograph better. There are two approaches to this mode: 1) using isocratic HILIC conditions or 2) using a sodium perchlorate gradient. The key to achieving HILIC conditions is to use greater than 70% organic solvent with the SCX column. Care should be taken to assure solubility of salts under these conditions.

### 4. Digestion of Isolated Polypeptides to Fragments

Digestion of polypeptide samples can be achieved using either enzymes or chemical means.

Cyanogen bromide (CNBr) may be used to digest polypeptide samples into fragments. For example, Washburn et al. (Nature Biotechnology 19: 242-7) describe CNBr digestion of insoluble fractions containing membrane polypeptides by incubating the fraction for 5 minutes at room temperature with 90% formic acid, followed by CNBr incubation overnight in dark. The same method can also be adapted for use in the instant invention. A potential drawback of the method is that some undesirable side-reactions or modifications of the resulting peptides may be present in the reaction mixture. For example, some peptide fragments may be oxidized or formylated (due to the presence of formic acid in the reaction), thus unnecessarily increasing the complexity of the subsequent analysis. In addition, toxicity of the reagent is another concern.

On the other hand, trypsin is highly robust and specific. In addition, it has the added benefit of cleaving behind Lys or Arg, thereby generating mostly doubly-charged polypeptide fragments which are most desirably for MS analysis, particularly for peptide sequencing using tandem mass-spectrometry. Therefore, the most preferred enzyme for digestion is Trypsin. However, a number of other enzymes sharing similar property and specifications, i.e. preferential cleavage after Lys or Arg, are well-known in the art. Those enzymes (see pp314-320, Enzyme Nomenclature, 1978, Academic Press, New York) can also be used to achieve similar results. Finally, in theory, other peptidases with different specificity may also be proper.

A combination of enzymatic digestion and chemical digestion can also be used if desired.

### 5. Fractionation Methods

Unbiased extraction of membrane polypeptides from biological samples is most readily accomplished by including detergents in extraction buffer. However, the very presence of detergent in samples will suppress the signals in subsequent mass-spectrometry analysis. Therefore, a fractionation step following membrane polypeptide isolation and digestion is desirable for the purpose of removing detergents and expanding the dynamic range of subsequent mass-spectrometry analysis by reducing the peptide complexity observed in each fraction.

In a preferred embodiment, strong cation exchange chromatography is used to fractionate peptide fragments following digestion, although other means of ion exchange chromatography may also employed under different circumstances to achieve the same goals of removing detergents and/or reducing sample complexity.

Traditionally, fractionation of undigested polypeptide samples are performed by electrophoresis, in which a supporting gel-matrix is used to separate polypeptides based on their size. Different variations of the basis methods have been developed to isolate polypeptide samples based on other properties, such as charge and isoelectric point (pI). These procedures are laborious and slow. In addition, relatively large amount of samples are required. For membrane polypeptides, these gel-based methods are particularly ill-suited for reasons mentioned before. Those problems have significantly limited their use in large scale high throughput analysis which is routine in current proteomic studies.

To meet the need of such studies, gel-free approaches have been employed to achieve fast, efficient separation, often with the added advantage of using small amount of samples. This feature is also ideal for direct connection with downstream sample analysis using mass spectrometry, particularly when used in combination with electrospray ionization (ESI).

In a preferred embodiment, nanoHPLC is used for peptide separation. Among the advantages of fast nano high-performance liquid chromatography (nanoHPLC) are consumption of small sample volume, mobile phase economy, and high through-put. Other separation means can also be used. For example, standard probe, a nanoflow source or especially capillary electrophoresis (CE) directly coupled are all possible alternatives.

### 6. Analytical Methods

In a preferred embodiment of the invention, nanoBPLC/μESI/FTMS is used for analyzing fractionated peptide samples. The nanoHPLC/μESI combination allows direct coupling of HPLC separation of peptide fragments with MS analysis. The high resolution and large dynamic range intrinsic to FTMS data is most desired in generating a peptide list for further analysis.

Fourier-transform ion cyclotron resonance (FTMS) offers distinct advantages, including high resolution, high mass accuracy, and high dynamic range. First introduced in 1974 by Comisarow and Marshall, FTMS is based on the principle of a charged particle orbiting in the presence of a magnetic field. While the ions are orbiting, a radio frequency (RF) signal is used to excite them and as a result of this RF excitation, the ions produce a detectable image current. The time-dependent image current can then be Fourier transformed to obtain the component frequencies of the different ions which correspond to their m/z.

Coupled to ESI and MALDI, FTMS can offer high accuracy with errors as low as ±0.001%, although such high mass accuracy is typically not necessary for our online detection analysis.

Other analytical methods may also be employed. For example, it is possible to modify an existing Time-of-Flight (TOF) Mass Spectrometer and adapt it for the same analysis. A time-offlight (TOF) analyzer is one of the simplest mass analyzing devices and is commonly used with MALDI ionization. Time-of-flight analysis is based on accelerating a set of ions to a detector with the same amount of energy. Because the ions have the same energy, yet a different mass, the ions reach the detector at different times. The smaller ions reach the detector first because of their greater velocity and the larger ions take longer, thus the analyzer is called time-of-flight because the mass is determined from the ions time of arrival. The arrival time of an ion at the detector is dependent upon the mass, charge, and kinetic energy of the ion. Since kinetic energy (KE) is equal to ½ mv2 or velocity v=(2KE/m)½, ions will travel a given distance, d, within a time, t, where t is dependent upon their m/z. However, typical TOF-MS may not provide the same large dynamic range offered by FTMS.

These analytical methods will yield highly accurate measurements of peptide mass-to-charge ratio. Coupled with retention time of each peptide fragment obtained from separation methods, these data can be deconvoluted and used to generate a list of all detected peptide fragments for each given sample so that the relative abundance of each peptide fragment can be compared across samples. These analyses can be done manually. Alternatively, computer algorithms that compile lists of deconvoluted masses with corresponding retention time information and compare two or more such lists can be developed, thereby significantly improving the speed of analysis.

### 7. Sequence Identification

Peptides or peptide fragments which abundance are significantly altered following certain treatments can then be identified by a variety of methods.

Targeted MS/MS with an LCQ ion trap mass spectrometer is a preferred method for peptide sequencing. The commercially available LCQ ion trap MS offers good sensitivity for the purpose of peptide sequencing.

In an ion trap the ions are trapped in a radio frequency quadrupole field. One method of using an ion trap for mass spectrometry is to generate ions externally with ESI or MALDI, using ion optics for sample injection into the trapping volume. The quadrupole ion trap typically consists of a ring electrode and two hyperbolic endcap electrodes. The motion of the ions trapped by the electric field resulting from the application of RF and DC voltages allows ions to be trapped or ejected from the ion trap. In the normal mode the RF is scanned to higher voltages, the trapped ions with the lowest m/z and are ejected through small holes in the endcap to a detector (a mass spectrum is obtained by resonantly exciting the ions and thereby ejecting from the trap and detecting them). As the RF is scanned further, higher m/z ratios become are ejected and detected. It is also possible to isolate one ion species by ejecting all others from the trap. The isolated ions can subsequently be fragmented by collisional activation (CAD/CID) and the fragments detected. The primary advantages of quadrupole ion traps is that multiple collision-induced dissociation experiments can be performed without having multiple analyzers. Other important advantages include its compact size, and the ability to trap and accumulate ions to increase the signal-to-noise ratio of a measurement. Quadrupole ion traps have been utilized in many applications predominantly including electrospray ionization MS/MS experiments on peptides and small molecules.

Peptide fragmentation patterns may be correlated to predicted peptide fragmentation patterns of peptides in polypeptide or nucleotide databases. Commercial software is available for this comparison such as Sequest (Finningan, San Jose, Calif.) or MASCOT (Manchester, England). Spectra not obtaining a match to known peptide sequences may be manually sequenced. The identified peptide sequences can then be searched against polypeptide or nucleotide databases, such as SwissProt or GeneBank, using BLAST method or a modified version thereof to identify the polypeptide source. In case that there is no exact match of the derived peptide sequence to any known sequences, there are a variety of “pattern matching” or “approximate string matching” algorithms known in the art which can be readily adapted for use in the present invention.

For instance, similarity tools developed by Needelman & Wunch (J. Mol Biol. 48:444-453, 1970) and Sellers (SIAM J Appl Math. 26:787-793, 1974) can be used to calculate a global similarity score between the entire lengths of the sequences being compared. This type of algorithm is not sensitive for highly diverged sequences, but does not need to be so in most embodiments of the present method. Another available method focuses on shorter regions of local similarity. Examples of local similarity algorithms include the Smith-Waterman (J Mol Biol 147:195-197, 1981), BLAST (Altschul et al, J Mol Biol 215:403-410, 1990), and FASTA (Pearson and Lipman, PNAS 85:2444-2448, 1988).

In certain embodiments, the subject method may use a string matching method based on bit operations or on arithmetic, rather than character comparisons. Some of the examples are the Shift-And method, Karp-Rabin fingerprint method, or the algorithm of Commentz-Walter (“A string matching algorithm fast on the average” Proc. 6th International Colloquium on Automata, Languages, and Programming (1979), pp. 118-132), which combines the Boyer-Moore technique with the Aho algorithm.

### 8. Uses in Proteomics

Mass spectrometry has emerged as a central technique in a wide variety of functional genomics, or proteomics approaches to study gene function in the post-genomics world. Mass spectrometric instrumentation continues to become more powerful and novel instrumental concepts are being put into use. The subject genomic searching system can be used as part of a proteomics discovery method.

For instance, the subject method can use peptide sequence information obtained by mass spectrometry as the identification method in “expression proteomics”, sequencing data from two or more different biological states/samples. Especially membrane fractions of two or more different biological states/samples.

Several interesting approaches have been taken recently towards the analysis of the proteome without the use of gel electrophoresis. In one such approach, the polypeptide population is separated by a variant of capillary electrophoresis and the intact polypeptides are then eluted into a Fourier transform ion cyclotron resonance mass spectrometer (FTMS). The FTMS is capable of storing the ions and measuring them at extremely high resolution and mass accuracy using a frequency based method. Measurement of several hundreds polypeptide components from lysates of Escherichia coli or yeast has already been shown. Jensen et al. (1999) Anal. Chem. 71:2076. Using a variant of the tandem mass spectrometric method, it may also be possible to identify the polypeptides “on-line” as they elute into the mass spectrometer. See, for example, Mørtz et al. (1996) PNAS 93:8264-8267; and Li et al. (1999) Anal. Chem. 71:4397.

In another approach, crude polypeptide mixtures such as those isolated from the membrane portion of the sample are digested, either in solution or as pellet. The resulting peptide mixture is then separated and analyzed by the LC/MS method outlined above. See Yates et al. (1997) Protein Chem. 16:495; and Link et al. (1999) Nat. Biotechnol. 17:676. As the capacity of the mass spectrometer to sequence co-eluting peptides increases, more and more complex polypeptide mixtures can be analyzed.

### 9. Business Methods

Yet another aspect of the present invention relates to a method of conducting a pharmaceutical business, comprising:

(i) by the above-described method, determining the identity of a target polypeptide isolated on the basis of the polypeptide being (a) having a differential cellular localization of interest, (b) having a differential expression pattern of interest,

(ii) identifying compounds by their ability to alter the abundance or subcellular localization of the target polypeptide;

(iii) conducting therapeutic profiling of compounds identified in step (ii), or further analogs thereof, for efficacy and toxicity in animals; and,

(iv) formulating a pharmaceutical preparation including one or more compounds identified in step (iii) as having an acceptable therapeutic profile.

The subject business method can include the additional step of establishing a distribution system for distributing the pharmaceutical preparation for sale, and may optionally include establishing a sales group for marketing the pharmaceutical preparation.

Still another aspect of the present invention provides a method of conducting a pharmaceutical business, comprising:

(i) by the above-described method, determining the identity of a target polypeptide isolated on the basis of the polypeptide: (a) having a differential cellular localization of interest, (b) having a differential expression pattern of interest;

(ii) (optionally) conducting therapeutic profiling of the target gene for efficacy and toxicity in animals; and

(iii) licensing, to a third party, the rights for further drug development of inhibitors or activators of the target gene.

### 10. Compound Library

A. Variegated Peptide Display

The invention provides a method to identify a compound that can alter the abundance of a specific target membrane polypeptide in a sample following treatment by the compound. The compound can be selected from a number of different libraries, such as a small molecule chemical compound library, a polypeptide library, or a peptidylmemetic library.

The variegated peptide libraries of the subject method can be generated by any of a number of methods, and, though not limited by, preferably exploit recent trends in the preparation of chemical libraries. The library can be prepared, for example, by either synthetic or biosynthetic approaches, and screened for activity against the D-enantiomer target in a variety of assay formats. As used herein, “variegated” refers to the fact that a population of peptides is characterized by having a peptide sequence which differ from one member of the library to the next. For example, in a given peptide library of N amino acids in length, the total number of different peptide sequences in the library is given by the product of Πnᵢ, wherein each nᵢ represents the number of different amino acid residues occurring at position i of the peptide. In a preferred embodiment of the present invention, the peptide display collectively produces a peptide library including at least 96 to 10⁷ different peptides, so that diverse peptides may be simultaneously assayed for the ability to interact with the target polypeptide.

Peptide libraries are systems which simultaneously display, in a form which permits interaction with a target polypeptide, a highly diverse and numerous collection of peptides. These peptides may be presented in solution (Houghten (1992) Biotechniques 13:412-421), or on beads (Lam (1991) Nature 354:82-84), chips (Fodor (1993) Nature 364:555-556), bacteria (Ladner U.S. Ser. No. 5,223,409), spores (Ladner U.S. Ser. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; and Ladner U.S. Ser. No. '409).

In one embodiment, the peptide library is derived to express a combinatorial library of peptides which are not based on any known sequence, nor derived from cDNA. That is, the sequences of the library are largely random. It will be evident that the peptides of the library may range in size from dipeptides to large polypeptides.

In another embodiment, the peptide library is derived to express a combinatorial library of peptides which are based at least in part on a known polypeptide sequence or a portion thereof (not a cDNA library). That is, the sequences of the library is semi-random, being derived by combinatorial mutagenesis of a known sequence(s). See, for example, Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992) PNAS 89:4457-4461. Accordingly, polypeptide(s) which are known ligands for a target polypeptide can be mutagenized by standard techniques to derive a variegated library of polypeptide sequences which can further be screened for agonists and/or antagonists.

In still another embodiment, the combinatorial polypeptides are produced from a cDNA library.

Depending on size, the combinatorial peptides of the library can be generated as is, or can be incorporated into larger fusion polypeptides. The fusion polypeptide can provide, for example, stability against degradation or denaturation, as well as a secretion signal if secreted. In an exemplary embodiment, the polypeptide library is provided as part of thioredoxin fusion polypeptides (see, for example, U.S. Pat. Nos. 5,270,181 and 5,292,646; and PCT publication WO94/02502). The combinatorial peptide can be attached on the terminus of the thioredoxin polypeptide, or, for short peptide libraries, inserted into the so-called active loop.

In preferred embodiments, the combinatorial polypeptides are in the range of 3-100 amino acids in length, more preferably at least 5-50, and even more preferably at least 10, 13, 15, 20 or 25 amino acid residues in length. Preferably, the polypeptides of the library are of uniform length. It will be understood that the length of the combinatorial peptide does not reflect any extraneous sequences which may be present in order to facilitate expression, e.g., such as signal sequences or invariant portions of a fusion polypeptide.

i) Biosynthetic Peptide Libraries

The harnessing of biological systems for the generation of peptide diversity is now a well established technique which can be exploited to generate the peptide libraries of the subject method. The source of diversity is the combinatorial chemical synthesis of mixtures of oligonucleotides. Oligonucleotide synthesis is a well-characterized chemistry that allows tight control of the composition of the mixtures created. Degenerate DNA sequences produced are subsequently placed into an appropriate genetic context for expression as peptides.

There are two principal ways in which to prepare the required degenerate mixture. In one method, the DNAs are synthesized a base at a time. When variation is desired at a base position dictated by the genetic code a suitable mixture of nucleotides is reacted with the nascent DNA, rather than the pure nucleotide reagent of conventional polynucleotide synthesis. The second method provides more exact control over the amino acid variation. First, trinucleotide reagents are prepared, each trinucleotide being a codon of one (and only one) of the amino acids to be featured in the peptide library. When a particular variable residue is to be synthesized, a mixture is made of the appropriate trinucleotides and reacted with the nascent DNA. Once the necessary “degenerate” DNA is complete, it must be joined with the DNA sequences necessary to assure the expression of the peptide, as discussed in more detail below, and the complete DNA construct must be introduced into the cell.

Whatever the method may be for generating diversity at the codon level, chemical synthesis of a degenerate gene sequence can be carried out in an automatic DNA synthesizer, and the synthetic genes can then be ligated into an appropriate gene for expression. The purpose of a degenerate set of genes is to provide, in one mixture, all of the sequences encoding the desired set of potential test peptide sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other polypeptides (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Because the number of different peptides one can create by this combination approach can be huge, and because the expectation is that peptides with the appropriate structural characteristics to serve as ligands for a given target polypeptide will be rare in the total population of the library, the need for methods capable of conveniently screening large numbers of clones is apparent. Several strategies for selecting peptide ligands from the library have been described in the art and are applicable to certain embodiments of the present method.

In one embodiment, a variegated peptide library can be expressed by a population of display packages to form a peptide display library. With respect to the display package on which the variegated peptide library is manifest, it will be appreciated from the discussion provided herein that the display package will often preferably be able to be (i) genetically altered to encode a test peptide, (ii) maintained and amplified in culture, (iii) manipulated to display the peptide in a manner permitting the peptide to interact with a target polypeptide during an affinity separation step, and (iv) affinity separated while retaining the peptide-encoding gene such that the sequence of the peptide can be obtained. In preferred embodiments, the display remains viable after affinity separation.

Ideally, the display package comprises a system that allows the sampling of very large variegated peptide display libraries, rapid sorting after each affinity separation round, and easy isolation of the peptide-encoding gene from purified display packages. The most attractive candidates for this type of screening are prokaryotic organisms and viruses, as they can be amplified quickly, they are relatively easy to manipulate, and large number of clones can be created. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages.

In addition to commercially available kits for generating phage display libraries (e.g. the Pharmacia Recombinant Phage Peptide System, catalog no. 27-9400-01; and the Stratagene SurfZAPTM phage display kit, catalog no. 240612), examples of methods and reagents particularly amenable for use in generating the variegated peptide display library of the present method can be found in, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; the Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

When the display is based on a bacterial cell, or a phage which is assembled periplasmically, the display means of the package will comprise at least two components. The first component is a secretion signal which directs the recombinant peptide to be localized on the extracellular side of the cell membrane (of the host cell when the display package is a phage). This secretion signal is characteristically cleaved off by a signal peptidase to yield a processed, “mature” peptide. The second component is a display anchor polypeptide which directs the display package to associate the peptide with its outer surface. As described below, this anchor polypeptide can be derived from a surface or coat polypeptide native to the genetic package.

When the display package is a bacterial spore, or a phage whose polypeptide coating is assembled intracellularly, a secretion signal directing the peptide to the inner membrane of the host cell is unnecessary. In these cases, the means for arraying the variegated peptide library comprises a derivative of a spore or phage coat polypeptide amenable for use as a fusion polypeptide.

In the instance wherein the display package is a phage, the cloning site for the test peptide sequences in the phagemid should be placed so that it does not substantially interfere with normal phage function. One such locus is the intergenic region as described by Zinder and Boeke, (1982) Gene 19:1-10. In an illustrative embodiment comprising an M13 phage display library, the test peptide sequence is preferably expressed at an equal or higher-level than the HL-cpIII product (described below) to maintain a sufficiently high VL concentration in the periplasm and provide efficient assembly (association) of VL with VH chains. For instance, a phagemid can be constructed to encode, as separate genes, both a VH/coat fusion polypeptide and a VL chain. Under the appropriate induction, both chains are expressed and allowed to assemble in the periplasmic space of the host cell, the assembled peptide being linked to the phage particle by virtue of the VH chain being a portion of a coat polypeptide fusion construct.

The number of possible peptides for a given library may, in certain instances, exceed 1012. To sample as many combinations as possible depends, in part, on the ability to recover large numbers of transformants. For phage with plasmid-like forms (as filamentous phage), electrotransformation provides an efficiency comparable to that of phage-transfection with in vitro packaging, in addition to a very high capacity for DNA input. This allows large amounts of vector DNA to be used to obtain very large numbers of transformants. The method described by Dower et al. (1988) Nucleic Acids Res., 16:6127-6145, for example, may be used to transform fd-tet derived recombinants at the rate of about 107 transformants/ug of ligated vector into E. coli (such as strain MC1061), and libraries may be constructed in fd-tet B1 of up to about 3×108 members or more. Increasing DNA input and making modifications to the cloning protocol within the ability of the skilled artisan may produce increases of greater than about 10-fold in the recovery of transformants, providing libraries of up to 1010 or more recombinants.

As will be apparent to those skilled in the art, in embodiments wherein high affinity peptides are sought, an important criteria for the present selection method can be that it is able to discriminate between peptides of different affinity for a particular target, and preferentially enrich for the peptides of highest affinity. Applying the well known principles of affinity and valence, it is understood that manipulating the display package to be rendered effectively monovalent can allow affinity enrichment to be carried out for generally higher binding affinities (i.e. binding constants in the range of 106 to 1010 M-1) as compared to the broader range of affinities isolable using a multivalent display package. To generate the monovalent display, the natural (i.e. wild-type) form of the surface or coat polypeptide used to anchor the peptide to the display can be added at a high enough level that it almost entirely eliminates inclusion of the peptide fusion polypeptide in the display package. Thus, a vast majority of the display packages can be generated to include no more than one copy of the peptide fusion polypeptide (see, for example, Garrad et al. (1991) Bio/Technology 9:1373-1377). In a preferred embodiment of a monovalent display library, the library of display packages will comprise no more than 5 to 10% polyvalent displays, and more preferably no more than 2% of the display will be polyvalent, and most preferably, no more than 1% polyvalent display packages in the population. The source of the wild-type anchor polypeptide can be, for example, provided by a copy of the wild-type gene present on the same construct as the peptide fusion polypeptide, or provided by a separate construct altogether.

a) Phage As Display Packages

Bacteriophage are attractive prokaryotic-related organisms for use in the subject method. Bacteriophage are excellent candidates for providing a display system of the variegated peptide library as there is little or no enzymatic activity associated with intact mature phage, and because their genes are inactive outside a bacterial host, rendering the mature phage particles metabolically inert. In general, the phage surface is a relatively simple structure. Phage can be grown easily in large numbers, they are amenable to the practical handling involved in many potential mass screening programs, and they carry genetic information for their own synthesis within a small, simple package. As the peptide gene is inserted into the phage genome, choosing the appropriate phage to be employed in the subject method will generally depend most on whether (i) the genome of the phage allows introduction of the peptide-encoding gene either by tolerating additional genetic material or by having replaceable genetic material; (ii) the virion is capable of packaging the genome after accepting the insertion or substitution of genetic material; and (iii) the display of the peptide on the phage surface does not disrupt virion structure sufficiently to interfere with phage propagation.

One concern presented with the use of phage is that the morphogenetic pathway of the phage determines the environment in which the peptide will have opportunity to fold. Periplasmically assembled phage are preferred as the displayed antibodies where the test peptide contains essential disulfides. However, in certain embodiments in which the display package forms intracellularly (e.g., where 1 phage are used), it has been demonstrated that the peptide may assume proper folding after the phage is released from the cell.

Another concern related to the use of phage, but also pertinent to the use of bacterial cells and spores as well, is that multiple infections could generate hybrid displays that carry the gene for one particular peptide yet have at least one or more different test peptides on their surfaces. Therefore, it can be preferable, though optional, to minimize this possibility by infecting cells with phage under conditions resulting in a low multiple infection. However, there may be circumstances in which high multiple-infection conditions would be desirable, such as to increase homologous recombination events between gene constructs encoding the peptide display in order to further expand the repertoire of the peptide display library.

For a given bacteriophage, the preferred display means is a polypeptide that is present on the phage surface (e.g. a coat polypeptide). Filamentous phage can be described by a helical lattice; isometric phage, by an icosahedral lattice. Each monomer of each major coat polypeptide sits on a lattice point and makes defined interactions with each of its neighbors. Polypeptides that fit into the lattice by making some, but not all, of the normal lattice contacts are likely to destabilize the virion by aborting formation of the virion as well as by leaving gaps in the virion so that the nucleic acid is not protected. Thus in bacteriophage, unlike the cases of bacteria and spores, it is generally important to retain in the peptide fusion polypeptides those residues of the coat polypeptide that interact with other polypeptides in the virion. For example, when using the M13 cpVIII polypeptide, the entire mature polypeptide will generally be retained with the peptide fragment being added to the N-terminus of cpVIII, while on the other hand it can suffice to retain only the last 100 carboxy terminal residues (or even fewer) of the M13 cpIII coat polypeptide in the peptide fusion polypeptide.

Under the appropriate induction, the peptide library is expressed and allowed to assemble in the bacterial cytoplasm, such as when the 1 phage is employed. The induction of the polypeptide(s) may be delayed until some replication of the phage genome, synthesis of some of the phage structural-polypeptides, and assembly of some phage particles has occurred. The assembled polypeptide chains then interact with the phage particles via the binding of the anchor polypeptide on the outer surface of the phage particle. The cells are lysed and the phage bearing the library-encoded test peptides (that correspond to the specific library sequences carried in the DNA of that phage) are released and isolated from the bacterial debris.

To enrich for and isolate phage which contain cloned library sequences that encode a desired polypeptide, and thus to ultimately isolate the nucleic acid sequences themselves, phage harvested from the bacterial debris are, for example, affinity purified. As described below, when a peptide which specifically binds a particular target polypeptide is desired, the target polypeptide can be used to retrieve phage displaying the desired peptide. The phage so obtained may then be amplified by infecting into host cells. Additional rounds of affinity enrichment followed by amplification may be employed until the desired level of enrichment is reached.

The enriched peptide-phage can also be screened with additional detection-techniques such as expression plaque (or colony) lift (see, e.g., Young and Davis, Science (1983) 222:778-782) whereby a labeled target polypeptide is used as a probe. The phage obtained from the screening protocol are infected into cells, propagated, and the phage DNA isolated and sequenced, and/or recloned into a vector intended for gene expression in prokaryotes or eukaryotes to obtain larger amounts of the particular peptide selected.

In yet another embodiment, the peptide is also transported to an extra-cytoplasmic compartment of the host cell, such as the bacterial periplasm, but as a fusion polypeptide with a viral coat polypeptide. In this embodiment the desired polypeptide (or one of its polypeptide chains if it is a multichain peptide) is expressed fused to a viral coat polypeptide which is processed and transported to the cell inner membrane. Other chains, if present, are expressed with a secretion leader and thus are also transported to the periplasm or other intracellular by extra-cytoplasmic location. The chains present in the extra-cytoplasm then assemble into a complete test peptide. The assembled molecules become incorporated into the phage by virtue of their attachment to the phage coat polypeptide as the phage extrude through the host membrane and the coat polypeptides assemble around the phage DNA. The phage bearing the test peptide may then be screened by affinity enrichment as described below.

1) Filamentous Phage

Filamentous bacteriophages, which include M13, fl, fd, Ifl, Ike, Xf, Pf1, and Pf3, are a group of related viruses that infect bacteria. They are termed filamentous because they are long, thin particles comprised of an elongated capsule that envelopes the deoxyribonucleic acid (DNA) that forms the bacteriophage genome. The F pili filamentous bacteriophage (Ff phage) infect only gram-negative bacteria by specifically adsorbing to the tip of F pili, and include fd, fl and M13.

Compared to other bacteriophage, filamentous phage in general are attractive for generating the peptide libraries of the subject method, and M13 in particular is especially attractive because: (i) the 3-D structure of the virion is known; (ii) the processing of the coat polypeptide is well understood; (iii) the genome is expandable; (iv) the genome is small; (v) the sequence of the genome is known; (vi) the virion is physically resistant to shear, heat, cold, urea, guanidinium chloride, low pH, and high salt; (vii) the phage is a sequencing vector so that sequencing is especially easy; (viii) antibiotic-resistance genes have been cloned into the genome with predictable results (Hines et al. (1980) Gene 11:207-218); (ix) it is easily cultured and stored, with no unusual or expensive media requirements for the infected cells, (x) it has a high burst size, each infected cell yielding 100 to 1000 M13 progeny after infection; and (xi) it is easily harvested and concentrated (Salivar et al. (1964) Virology 24: 359-371). The entire life cycle of the filamentous phage M13, a common cloning and sequencing vector, is well understood. The genetic structure of M13 is well known, including the complete sequence (Schaller et al. in The Single-Stranded DNA Phages eds. Denhardt et al. (NY: CSHL Press, 1978)), the identity and function of the ten genes, and the order of transcription and location of the promoters, as well as the physical structure of the virion (Smith et al. (1985) Science 228:1315-1317; Raschad et al. (1986) Microbiol Dev 50:401-427; Kuhn et al. (1987) Science 238:1413-1415; Zimmerman et al. (1982) J Biol Chem 257:6529-6536; and Banner et al. (1981) Nature 289:814-816). Because the genome is small (6423 bp), cassette mutagenesis is practical on RF M13 (Current Protocols in Molecular Biology, eds. Ausubel et al. (NY: John Wiley & Sons, 1991)), as is single-stranded oligonucleotide directed mutagenesis (Fritz et al. in DNA Cloning, ed by Glover (Oxford, UK: IRC Press, 1985)). M13 is a plasmid and transformation system in itself, and an ideal sequencing vector. M13 can be grown on Rec? strains of E. coli. The M13 genome is expandable (Messing et al. in The Single-Stranded DNA Phages, eds Denhardt et al. (NY: CSHL Press, 1978) pages 449-453; and Fritz et al., supra) and M13 does not lyse cells. Extra genes can be inserted into M13 and will be maintained in the viral genome in a stable manner.

The mature capsule or Ff phage is comprised of a coat of five phage-encoded gene products: cpVIII, the major coat polypeptide product of gene VIII that forms the bulk of the capsule; and four minor coat polypeptides, cpIII and cpIV at one end of the capsule and cpVII and cpIX at the other end of the capsule. The length of the capsule is formed by 2500 to 3000 copies of cpVIII in an ordered helix array that forms the characteristic filament structure. The gene III-encoded polypeptide (cpIII) is typically present in 4 to 6 copies at one end of the capsule and serves as the receptor for binding of the phage to its bacterial host in the initial phase of infection. For detailed reviews of Ff phage structure, see Rasched et al., Microbiol. Rev., 50:401?427 (1986); and Model et al., in The Bacteriophages, Volume 2, R. Calendar, Ed., Plenum Press, pp. 375?456 (1988).

The phage particle assembly involves extrusion of the viral genome through the host cell's membrane. Prior to extrusion, the major coat polypeptide cpVIII and the minor coat polypeptide cpIII are synthesized and transported to the host cell's membrane. Both cpVIII and cpIII are anchored in the host cell membrane prior to their incorporation into the mature particle. In addition, the viral genome is produced and coated with cpV polypeptide. During the extrusion process, cpV-coated genomic DNA is stripped of the cpV coat and simultaneously recoated with the mature coat polypeptides.

Both cpIII and cpVIII polypeptides include two domains that provide signals for assembly of the mature phage particle. The first domain is a secretion signal that directs the newly synthesized polypeptide to the host cell membrane. The secretion signal is located at the amino terminus of the polypeptide and targets the polypeptide at least to the cell membrane. The second domain is a membrane anchor domain that provides signals for association with the host cell membrane and for association with the phage particle during assembly. This second signal for both cpVIII and cpIII comprises at least a hydrophobic region for spanning the membrane.

The 50 amino acid mature gene VIII coat polypeptide (cpVIII) is synthesized as a 73 amino acid precoat (Ito et al. (1979) PNAS 76:1199-1203). The cpVIII polypeptide has been extensively studied as a model membrane polypeptide because it can integrate into lipid bilayers such as the cell membrane in an asymmetric orientation with the acidic amino terminus toward the outside and the basic carboxy terminus toward the inside of the membrane. The first 23 amino acids constitute a typical signal-sequence which causes the nascent polypeptide to be inserted into the inner cell membrane. An E. coli signal peptidase (SP?I) recognizes amino acids 18, 21, and 23, and, to a lesser extent, residue 22, and cuts between residues 23 and 24 of the precoat (Kuhn et al. (1985) J. Biol. Chem. 260:15914-15918; and Kuhn et al. (1985) J. Biol. Chem. 260:15907-15913). After removal of the signal sequence, the amino terminus of the mature coat is located on the periplasmic side of the inner membrane; the carboxy terminus is on the cytoplasmic side. About 3000 copies of the mature coat polypeptide associate side-by-side in the inner membrane.

The sequence of gene VIII is known, and the amino acid sequence can be encoded on a synthetic gene. Mature gene VIII polypeptide makes up the sheath around the circular ssDNA. The gene VIII polypeptide can be a suitable anchor polypeptide because its location and orientation in the virion are known (Banner et al. (1981) Nature 289:814-816). Preferably, the test peptide is attached to the amino terminus of the mature M13 coat polypeptide to generate the phage display library. As set out above, manipulation of the concentration of both the wild-type cpVIII and test peptide/cpVIII fusion in an infected cell can be utilized to decrease the avidity of the display and thereby enhance the detection of high affinity antibodies directed to the target epitope(s).

Another vehicle for displaying the test peptide library is by expressing it as a domain of a chimeric gene containing part or all of gene III. When monovalent displays are required, expressing the test peptide as a fusion polypeptide with cpIII can be a preferred embodiment, as manipulation of the ratio of wild-type gpIII to chimeric cpIII during formation of the phage particles can be readily controlled. This gene encodes one of the minor coat polypeptides of M13. In particular, the single-stranded circular phage DNA associates with about five copies of the gene III polypeptide and is then extruded through the patch of membrane-associated coat polypeptide in such a way that the DNA is encased in a helical sheath of polypeptide (Webster et al. in The Single-Stranded DNA Phages, eds Dressler et al. (NY:CSHL Press, 1978).

Manipulation of the sequence of cpIII has demonstrated that the C-terminal 23 amino acid residue stretch of hydrophobic amino acids normally responsible for a membrane anchor function can be altered in a variety of ways and retain the capacity to associate with membranes. Ff phage-based expression vectors were first described in which the cpIII amino acid residue sequence was modified by insertion of polypeptide “epitopes” (Parmely et al., Gene (1988) 73:305-318; and Cwirla et al., PNAS (1990) 87:6378?6382) or an amino acid residue sequence defining a larger polypeptide domain (McCafferty et al., Science (1990) 348:552?554). It has been demonstrated that insertions into gene III can result in the production of novel polypeptide domains on the virion outer surface. (Smith (1985) Science 228:1315-1317; and de la Cruz et al. (1988) J. Biol. Chem. 263:4318-4322). The test peptide-encoding gene may be fused to gene III at the site used by Smith and by de la Cruz et al., e.g., at a codon corresponding to another domain boundary or to a surface loop of the polypeptide, or to the amino terminus of the mature polypeptide.

Similar constructions could be made with other filamentous phage. Pf3 is a well known filamentous phage that infects Pseudomonas aerugenosa cells that harbor an IncP-I plasmid. The entire genome has been sequenced ((Luiten et al. (1985) J. Virol. 56:268-276) and the genetic signals involved in replication and assembly are known (Luiten et al. (1987) DNA 6:129-137). The major coat polypeptide of PF3 is unusual in having no signal peptide to direct its secretion. The sequence has charged residues ASP-7, ARG-37, LYS-40, and PHE44 which is consistent with the amino terminus being exposed. Thus, to cause a test peptide to appear on the surface of Pf3, a tripartite gene can be constructed which comprises a signal sequence known to cause secretion in P. aerugenosa, fused in-frame to a gene fragment encoding the test peptide sequence, which is fused in-frame to DNA encoding the mature Pf3 coat polypeptide. Optionally, DNA encoding a flexible linker of one to 10 amino acids is introduced between the test peptide fragment and the Pf3 coat-polypeptide gene. This tripartite gene is introduced into Pf3. Once the signal sequence is cleaved off, the test peptide is in the periplasm and the mature coat polypeptide acts as an anchor and phage-assembly signal.

2) Bacteriophage fX174

The bacteriophage fX174 is a very small icosahedral virus which has been thoroughly studied by genetics, biochemistry, and electron microscopy (see The Single Stranded DNA Phages (eds. Den hard et al. (NY:CSHL Press, 1978)). Three gene products of fX174 are present on the outside of the mature virion: F (cased), G (major spike polypeptide, 60 copies per virion), and H (minor spike polypeptide, 12 copies per virion). The G polypeptide comprises 175 amino acids, while H comprises 328 amino acids. The F polypeptide interacts with the single-stranded DNA of the virus. The polypeptides F, G, and H are translated from a single mRNA in the viral infected cells. As the virus is so tightly constrained because several of its genes overlap, fX174 is not typically used as a cloning vector due to the fact that it can accept very little additional DNA. However, mutations in the viral G gene (encoding the G polypeptide) can be rescued by a copy of the wild-type G gene carried on a plasmid that is expressed in the same host cell (Chambers et al. (1982) Nuc Acid Res 10:6465-6473). In one embodiment, one or more stop codons are introduced into the G gene so that no G polypeptide is produced from the viral genome. Nucleic acid encoding the variegated peptide library can then be fused with the nucleic acid sequence of the H gene. An amount of the viral G gene equal to the size of the test peptide gene fragment is eliminated from the fX174 genome, such that the size of the genome is ultimately unchanged. Thus, in host cells also transformed with a second plasmid expressing the wild-type G polypeptide, the production of viral particles from the mutant virus is rescued by the exogenous G polypeptide source. Where it is desirable that only one test peptide be displayed per *X174 particle (e.g., monovalent), the second plasmid can further include one or more copies of the wild-type H polypeptide gene so that a mix of H and test peptide/H polypeptides will be predominated by the wild-type H upon incorporation into phage particles.

3) Large DNA Phage

Phage such as 1 or T4 have much larger genomes than do M13 or fX174, and have more complicated 3-D capsid structures than M13 or fPX174, with more coat polypeptides to choose from. In embodiments of the invention whereby the peptide library is processed and assembled into a functional form and associates with the bacteriophage particles within the cytoplasm of the host cell, bacteriophage 1 and derivatives thereof are examples of suitable vectors. The intracellular morphogenesis of phage 1 can potentially prevent polypeptide domains that ordinarily contain disulfide bonds from folding correctly. However, variegated libraries expressing a population of functional antibodies, including both heavy and light chain variable regions, have been generated in 1 phage, indicating that disulfide bonds can be formed in the test peptide library. (Huse et al. (1989) Science 246:1275-1281; Mullinax et al. (1990) PNAS 87:8095-8099; and Pearson et al. (1991) PNAS 88:2432-2436). Such strategies take advantage of the rapid construction and efficient transformation abilities of 1 phage.

When used for expression of peptide sequences, library DNA sequences may be readily inserted into a 1 vector. For instance, variegated peptide libraries have been constructed by modification of 1 ZAP II (Short et al. (1988) Nuc Acid Res 16:7583) comprising inserting the peptide-encoding nucleic acid into the multiple cloning site of a 1 ZAP II vector (Huse et al. supra.).

b) Bacterial Cells as Display Packages

Recombinant peptides are able to cross bacterial membranes after the addition of bacterial leader sequences to the peptides (Better et al (1988) Science 240:1041-1043; and Skerra et al. (1988) Science 240:1038-1041). In addition, recombinant peptides have been fused to outer membrane polypeptides for surface presentation. Accordingly, one strategy for displaying test peptides on bacterial cells comprises generating a fusion protein by adding the test peptide to cell surface exposed portions of an integral outer membrane protein (Fuchs et al. (1991) Bio/Technology 9:1370-1372). In selecting a bacterial cell to serve as the display package, any well-characterized bacterial strain will typically be suitable, provided the bacteria may be grown in culture, engineered to display the peptide library on its surface, and is compatible with the particular affinity selection process practiced in the subject method. Among bacterial cells, the preferred display systems include Salmonella typhimurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and especially Escherichia coli. Many bacterial cell surface proteins useful in the present invention have been characterized, and works on the localization of these proteins and the methods of determining their structure include Benz et al. (1988) Ann Rev Microbiol 42: 359-393; Balduyck et al. (1985) Biol Chem Hoppe-Seyler 366:9-14; Ehrmann et al (1990) PNAS 87:7574-7578; Heijne et al. (1990) Protein Engineering 4:109-112; Ladner et al. U.S. Pat. No. 5,223,409; Ladner et al. WO88/06630; Fuchs et al. (1991) Bio/technology 9:1370-1372; and Goward et al. (1992) TIBS 18:136-140.

To further illustrate, the LamB protein of E. coli is a well understood surface protein that can be used to generate a variegated library of test peptides (see, for example, Ronco et al. (1990) Biochemie 72:183-189; van der Weit et al. (1990) Vaccine 8:269-277; Charabit et al. (1988) Gene 70:181-189; and Ladner U.S. Pat. No. 5,222,409). LamB of E. coli is a porin for maltose and maltodextrin transport, and serves as the receptor for adsorption of bacteriophages 1 and K10. LamB is transported to the outer membrane if a functional N-terminal signal sequence is present (Benson et al. (1984) PNAS 81:3830-3834). As with other cell surface proteins, LamB is synthesized with a typical signal-sequence which is subsequently removed. Thus, the variegated peptide-encoding gene library can be cloned into the LamB gene such that the resulting library of fusion proteins comprise a portion of LamB sufficient to anchor the protein to the cell membrane with the test peptide portion oriented on the extracellular side of the membrane. Secretion of the extracellular portion of the fusion protein can be facilitated by inclusion of the LamB signal sequence, or other suitable signal sequence, as the N-terminus of the protein.

The E. coli LamB has also been expressed in functional form in S. typhimurium (Harkki et al. (1987) Mol Gen Genet 209:607-611), V. cholerae (Harkki et al. (1986) Microb Pathol 1:283-288), and K. pneumonia (Wehmeier et al. (1989) Mol Gen Genet 215:529-536), so that one could display a population of test peptides in any of these species as a fusion to E. coli LamB. Moreover, K. pneumonia expresses a maltoporin similar to LamB which could also be used. In P. aeruginosa, the D1 protein (a homologue of LamB) can be used (Trias et al. (1988) Biochem Biophys Acta 938:493-496). Similarly, other bacterial surface proteins, such as PAL, OmpA, OmpC, OmpF, PhoE, pilin, BtuB, FepA, FhuA, IutA, FecA and FhuE, may be used in place of LamB as a portion of the display means in a bacterial cell.

c) Bacterial Spores as Display Packages

Bacterial spores also have desirable properties as display package candidates in the subject method. For example, spores are much more resistant than vegetative bacterial cells or phage to chemical and physical agents, and hence permit the use of a great variety of affinity selection conditions. Also, Bacillus spores neither actively metabolize nor alter the proteins on their surface. However, spores have the disadvantage that the molecular mechanisms that trigger sporulation are less well worked out than is the formation of M13 or the export of protein to the outer membrane of E. coli, though such a limitation is not a serious detractant from their use in the present invention.

Bacteria of the genus Bacillus form endospores that are extremely resistant to damage by heat, radiation, desiccation, and toxic chemicals (reviewed by Losick et al. (1986) Ann Rev Genet 20:625-669). This phenomenon is attributed to extensive intermolecular cross-linking of the coat proteins. In certain embodiments of the subject method, such as those which include relatively harsh affinity separation steps, such spores can be the preferred display package. Endospores from the genus Bacillus are more stable than are, for example, exospores from Streptomyces. Moreover, Bacillus subtilis forms spores in 4 to 6 hours, whereas Streptomyces species may require days or weeks to sporulate. In addition, genetic knowledge and manipulation is much more developed for B. subtilis than for other spore-forming bacteria.

Viable spores that differ only slightly from wild-type are produced in B. subtilis even if any one of four coat proteins is missing (Donovan et al. (1987) J Mol Biol 196: 1-10). Moreover, plasmid DNA is commonly included in spores, and plasmid encoded proteins have been observed on the surface of Bacillus spores (Debra et al. (1986) J Bacteriol 165:258-268). Thus, it can be possible during sporulation to express a gene encoding a chimeric coat protein comprising a test peptide of the variegated gene library, without interfering materially with spore formation.

To illustrate, several polypeptide components of B. subtilis spore coat (Donovan et al. (1987) J Mol Biol 196: 1-10) have been characterized. The sequences of two complete coat proteins and amino-terminal fragments of two others have been determined. Fusion of the test peptide sequence to cotC or cotD fragments is likely to cause the test peptide to appear on the spore surface. The genes of each of these spore coat proteins are preferred as neither cotC or cotD are post-translationally modified (see Lader et al. U.S. Pat. No. 5,223,409).

ii) Synthetic Peptide Libraries

In contrast to the recombinant methods, in vitro chemical synthesis provides a method for generating libraries of compounds, without the use of living organisms, that can be screened for ability to bind to a target protein. Although in vitro methods have been used for quite some time in the pharmaceutical industry to identify potential drugs, recently developed methods have focused on rapidly and efficiently generating and screening large numbers of compounds and are particularly amenable to generating peptide libraries for use in the subject method. The various approaches to simultaneous preparation and analysis of large numbers of synthetic peptides (herein “multiple peptide synthesis” or “MPS”) each rely on the fundamental concept of synthesis on a solid support introduced by Merrifield in 1963 (Merrifield, R. B. (1963) J Am Chem Soc 85:2149-2154; and references cited in section I above). Generally, these techniques are not dependent on the protecting group or activation chemistry employed, although most workers today avoid Merrifield's original tBoc/Bzl strategy in favor of the more mild Fmoc/tBu chemistry and efficient hydroxybenzotriazole-based coupling agents. Many types of solid matrices have been successfully used in MPS, and yields of individual peptides synthesized vary widely with the technique adopted (e.g., nanomoles to millimoles).

a) Multipin Synthesis

One form that the peptide library of the subject method can take is the multipin library format. Briefly, Geysen and co-workers (Geysen et al. (1984) PNAS 81:3998-4002) introduced a method for generating peptide by a parallel synthesis on polyacrylic acid-grated polyethylene pins arrayed in the microtitre plate format. In the original experiments, about 50 nmol of a single peptide sequence was covalently linked to the spherical head of each pin, and interactions of each peptide with receptor or antibody could be determined in a direct binding assay. The Geysen technique can be used to synthesize and screen thousands of peptides per week using the multipin method, and the tethered peptides may be reused in many assays. In subsequent work, the level of peptide loading on individual pins has been increased to as much as 2 *mol/pin by grafting greater amounts of functionalized acrylate derivatives to detachable pin heads, and the size of the peptide library has been increased (Valerio et al. (1993) Int J Pept Protein Res 42:1-9). Appropriate linker moieties have also been appended to the pins so that the peptides may be cleaved from the supports after synthesis for assessment of purity and evaluation in competition binding or functional bioassays (Bray et al. (1990) Tetrahedron Lett 31:5811-5814; Valerio et al. (1991) Anal Biochem 197:168-177; Bray et al. (1991) Tetrahedron Lett 32:6163-6166).

More recent applications of the multipin method of MPS have taken advantage of the cleavable linker strategy to prepare soluble peptides (Maeji et al. (1990) J Immunol Methods 134:23-33; Gammon et al. (1991) J Exp Med 173:609-617; Mutch et al. (1991) Pept Res 4:132-137).

b) Divide-Couple-Recombine

In yet another embodiment, a variegated library of peptides can provide on a set of beads utilizing the strategy of divide-couple-recombine (see, e.g., Houghten (1985) PNAS 82:5131-5135; and U.S. Pat. Nos. 4,631,211; 5,440,016; 5,480,971). Briefly, as the name implies, at each synthesis step where degeneracy is introduced into the library, the beads are divided into as many separate groups to correspond to the number of different amino acid residues to be added that position, the different residues coupled in separate reactions, and the beads recombined into one pool for the next step.

In one embodiment, the divide-couple-recombine strategy can be carried out using the so-called “tea bag” MPS method first developed by Houghten, peptide synthesis occurs on resin that is sealed inside porous polypropylene bags (Houghten et al. (1986) PNAS 82:5131-5135). Amino acids are coupled to the resins by placing the bags in solutions of the appropriate individual activated monomers, while all common steps such as resin washing and *-amino group deprotection are performed simultaneously in one reaction vessel. At the end of the synthesis, each bag contains a single peptide sequence, and the peptides may be liberated from the resins using a multiple cleavage apparatus (Houghten et al. (1986) Int J Pept Protein Res 27:673-678). This technique offers advantages of considerable synthetic flexibility and has been partially automated (Beck-Sickinger et al. (1991) Pept Res 4:88-94). Moreover, soluble peptides of greater than 15 amino acids in length can be produced in sufficient quantities (>500 *mol) for purification and complete characterization if desired.

Multiple peptide synthesis using the tea-bag approach is useful for the production of a peptide library, albeit of limited size, for screening the present method, as is illustrated by its use in a range of molecular recognition problems including antibody epitope analysis (Houghten.et al. (1986) PNAS 82:5131-5135), peptide hormone structure-function studies (Beck-Sickinger et al. (1990) Int J Pept Protein Res 36:522-530; Beck-Sickinger et al. (1990) Eur J Biochem 194:449-456), and protein conformational mapping (Zimmerman et al. (1991) Eur J Biochem 200:519-528).

An exemplary synthesis of a set of mixed peptides having equimolar amounts of the twenty natural amino acid residues is as follows. Aliquots of five grams (4.65 mmols) of p-methylbenzhydrylamine hydrochloride resin (MBHA) are placed into twenty porous polypropylene bags. These bags are placed into a common container and washed with 1.0 liter of CH2Cl2 three times (three minutes each time), then again washed three times (three minutes each time) with 1.0 liter of 5 percent DIEA/CH2Cl2 (DIEA=diisopropylethylamine; CH2C12=DCM). The bags are then rinsed with DCM and placed into separate reaction vessels each containing 50 ml (0.56M) of the respective t-BOC-amino acid/DCM. N,N-Diisopropylcarbodiimide (DIPCDI; 25 ml; 1.12M) is added to each container, as a coupling agent. Twenty amino acid derivatives are separately coupled to the resin in 50/50 (v/v) DMF/DCM. After one hour of vigorous shaking, Gisen's picric acid test (Gisen (1972) Anal. Chem. Acta 58:248-249) is performed to determine the completeness of the coupling reaction. On confirming completeness of reaction, all of the resin packets are then washed with 1.5 liters of DMF and washed two more times with 1.5 liters of CH2C12. After rinsing, the resins are removed from their separate packets and admixed together to form a pool in a common bag. The resulting resin mixture is then dried and weighed, divided again into 20 equal portions (aliquots), and placed into 20 further polypropylene bags (enclosed).

In a common reaction vessel the following steps are carried out: (1) deprotection is carried out on the enclosed aliquots for thirty minutes with 1.5 liters of 55 percent TFA/DCM; and 2) neutralization is carried out with three washes of 1.5 liters each of 5 percent DIEA/DCM. Each bag is placed in a separate solution of activated t-BOC-amino acid derivative and the coupling reaction carried out to completion as before. All coupling reactions are monitored using the above quantitative picric acid assay.

Next, the bags are opened and the resulting t-BOC-protected dipeptide resins are mixed together to form a pool, aliquots are made from the pool, the aliquots are enclosed, deprotected and further reactions are carried out. This process can be repeated any number of times yielding at each step an equimolar representation of the desired number of amino acid residues in the peptide chain. The principal process steps are conveniently referred to as a divide-couple-recombine synthesis.

After a desired number of such couplings and mixtures are carried out, the polypropylene bags are kept separated to here provide the twenty sets having the amino-terminal residue as the single, predetermined residue, with, for example, positions 2-4 being occupied by equimolar amounts of the twenty residues. To prepare sets having the single, predetermined amino acid residue at other than the amino-terminus, the contents of the bags are not mixed after adding a residue at the desired, predetermined position. Rather, the contents of each of the twenty bags are separated into 20 aliquots, deprotected and then separately reacted with the twenty amino acid derivatives. The contents of each set of twenty bags thus produced are thereafter mixed and treated as before-described until the desired oligopeptide length is achieved.

c) Multiple Peptide Synthesis through Coupling of Amino Acid Mixtures

Simultaneous coupling of mixtures of activated amino acids to a single resin support has been used as a multiple peptide synthesis strategy on several occasions (Geysen et al. (1986) Mol Immunol 23:709-715; Tjoeng et al. (1990) Int J Pept Protein Res 35:141-146; Rutter et al. (1991) U.S. Pat. No. 5,010,175; Birkett et al. (1991) Anal Biochem 196:137-143; Petithory et al. (1991) PNAS 88:11510-11514) and can have applications in the subject method. For example, four to seven analogs of the magainin 2 and angiotensinogen peptides were successfully synthesized and resolved in one HPLC purification after coupling a mixture of amino acids at a single position in each sequence (Tjoeng et al. (1990) Int J Pept Protein Res 35:141-146). This approach has also been used to prepare degenerate peptide mixtures for defining the substrate specificity of endoproteolytic enzymes (Birkett et al. (1991) Anal Biochem 196:137-143; Petithory et al. (1991) PNAS 88:11510-11514). In these experiments a series of amino acids was substituted at a single position within the substrate sequence. After proteolysis, Edman degradation was used to quantitate the yield of each amino acid component in the hydrolysis product and hence to evaluate the relative kcat/Km values for each substrate in the mixture.

However, it is noted that the operational simplicity of synthesizing many peptides by coupling monomer mixtures is offset by the difficulty in controlling the composition of the products. The product distribution reflects the individual; rate constants for the competing coupling reactions, with activated derivatives of sterically hindered residues such as valine or isoleucine adding at a significantly slower rate than glycine or alanine for example. The nature of the resin-bound component of the acylation reaction also influences the addition rate, and the relative rate constants for the formation of 400 dipeptides form the 20 genetically coded amino acids have been determined by Rutter and Santi (Rutter et al. (1991) U.S. Pat. No. 5,010,175). These reaction rates can be used to guide the selection of appropriate relative concentrations of amino acids in the mixture to favor more closely equimolar coupling yields.

d) Multiple Peptide Synthesis on Nontraditional Solid Supports

The search for innovative methods of multiple peptide synthesis has led to the investigation of alternative polymeric supports to the polystyrene-divinylbenzene matrix originally popularized by Merrifield. Cellulose, either in the form of paper disks (Blankemeyer-Menge et al. (1988) Tetrahedron Lett 29-5871-5874; Frank et al. (1988) Tetrahedron 44:6031-6040; Eichler et al. (1989) Collect Czech Chem Commun 54:1746-1752; Frank, R. (1993) Bioorg Med Chem Lett 3:425-430) or cotton fragments (Eichler et al. (1991) Pept Res 4:296-307; Schmidt et al. (1993) Bioorg Med Chem Lett 3:441-446) has been successfully functionalized for peptide synthesis. Typical loadings attained with cellulose paper range from 1 to 3 *mol/cm2, and HPLC analysis of material cleaved from these supports indicates a reasonable quality for the synthesized peptides. Alternatively, peptides may be synthesized on cellulose sheets via non-cleavable linkers and then used in ELISA-based binding studies (Frank, R. (1992) Tetrahedron 48:9217-9232). The porous, polar nature of this support may help suppress unwanted nonspecific protein binding effects. By controlling the volume of activated amino acids and other reagents spotted on the paper, the number of peptides synthesized at discrete locations on the support can be readily varied. In one convenient configuration spots are made in an 8×12 microtiter plate format. Frank has used this technique to map the dominant epitopes of an antiserum raised against a human cytomegalovirus protein, following the overlapping peptide screening (Pepscan) strategy of Geysen (Frank, R. (1992) Tetrahedron 48:9217-9232). Other membrane-like supports that may be used for multiple solid-phase synthesis include polystyrene-grafted polyethylene films (Berg et al. (1989) J Am Chem Soc 111:8024-8026).

e) Combinatorial Libraries by Light-Directed, Spatially Addressable Parallel Chemical Synthesis

A scheme of combinatorial synthesis in which the identity of a compound is given by its locations on a synthesis substrate is termed a spatially-addressable synthesis. In one embodiment, the combinatorial process is carried out by controlling the addition of a chemical reagent to specific locations on a solid support (Dower et al. (1991) Annu Rep Med Chem 26:271-280; Fodor, S. P. A. (1991) Science 251:767; Pirrung et al. (1992) U.S. Pat. No. 5,143,854; Jacobs et al. (1994) Trends Biotechnol 12:19-26). The technique combines two well-developed technologies: solid-phase peptide synthesis chemistry and photolithography. The high coupling yields of Merrifield chemistry allow efficient peptide synthesis, and the spatial resolution of photolithography affords miniaturization. The merging of these two technologies is done through the use of photolabile amino protecting groups in the Merrifield synthetic procedure.

The key points of this technology are illustrated in Gallop et al. (1994) J Med Chem 37:1233-1251. A synthesis substrate is prepared for amino acid coupling through the covalent attachment of photolabile nitroveratryloxycarbonyl (NVOC) protected amino linkers. Light is used to selectively activate a specified region of the synthesis support for coupling. Removal of the photolabile protecting groups by lights (deprotection) results in activation of selected areas. After activation, the first of a set of amino acids, each bearing a photolabile protecting group on the amino terminus, is exposed to the entire surface. Amino acid coupling only occurs in regions that were addressed by light in the preceding step. The solution of amino acid is removed, and the substrate is again illuminated through a second mask, activating a different region for reaction with a second protected building block. The pattern of masks and the sequence of reactants define the products and their locations. Since this process utilizes photolithography techniques, the number of compounds that can be synthesized is limited only by the number of synthesis sites that can be addressed with appropriate resolution. The position of each compound is precisely known; hence, its interactions with other molecules can be directly assessed. The target protein can be labeled with a fluorescent reporter group to facilitate the identification of specific interactions with individual members of the matrix.

In a light-directed chemical synthesis, the products depend on the pattern of illumination and on the order of addition of reactants. By varying the lithographic patterns, many different sets of test peptides can be synthesized in the same number of steps; this leads to the generated of many different masking strategies.

f) Encoded Combinatorial Libraries

In yet another embodiment, the subject method utilizes a peptide library provided with an encoded tagging system. A recent improvement in the identification of active compounds from combinatorial libraries employs chemical indexing systems using tags that uniquely encode the reaction steps a given bead has undergone and, by inference, the structure it carries. Conceptually, this approach mimics phage display libraries above, where activity derives from expressed peptides, but the structures of the active peptides are deduced from the corresponding genomic DNA sequence. The first encoding of synthetic combinatorial libraries employed DNA as the code. Two forms of encoding have been reported: encoding with sequenceable bio-oligomers (e.g., oligonucleotides and peptides), and binary encoding with non-sequenceable tags.

1) Tagging with Sequenceable Bio-oligomers

The principle of using oligonucleotides to encode combinatorial synthetic libraries was described in 1992 (Brenner et al. (1992) PNAS 89:5381-5383), and an example of such a library appeared the following year (Needles et al. (1993) PNAS 90:10700-10704). A combinatorial library of nominally 77 (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH2 groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following the such techniques, the peptide libraries can be derived for use in the subject method and screened using the D-enantiomer of the target protein.

It is noted that an alternative approach useful for generating nucleotide-encoded synthetic peptide libraries employs a branched linker containing selectively protected OH and NH2 groups (Nielsen et al. (1993) J Am Chem Soc 115:9812-9813; and Nielsen et al. (1994) Methods Compan Methods Enzymol 6:361-371). This approach requires that equimolar quantities of test peptide and tag co-exist, though this may be a potential complication in assessing biological activity, especially with nucleic acid based targets.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis on non-oligomeric libraries. In preferred embodiments, the libraries employ linkers permitting selective detachment of the test peptide library member for bioassay, in part (as described infra) because assays employing beads limit the choice of targets, and in part because the tags are potentially susceptible to biodegradation.

Peptides themselves have been employed as tagging molecules for combinatorial libraries. Two exemplary approaches are described in the art, both of which employ branched linkers to solid phase upon which coding and ligand strands are alternately elaborated. In the first approach (Kerr J M et al. (1993) J Am Chem Soc 115:2529-2531), orthogonality in synthesis is achieved by employing acid-labile protection for the coding strand and base-labile protection for the ligand strand.

In an alternative approach (Nikolaiev et al. (1993) Pept Res 6:161-170), branched linkers are employed so that the coding unit and the test peptide are both attached to the same functional group on the resin. In one embodiment, a linker can be placed between the branch point and the bead so that cleavage releases a molecule containing both code and ligand (Ptek et al. (1991) Tetrahedron Lett 32:3891-3894). In another embodiment, the linker can be placed so that the test peptide can be selectively separated from the bead, leaving the code behind. This last construct is particularly valuable because it permits screening of the test peptide without potential interference, or biodegradation, of the coding groups. Examples in the art of independent cleavage and sequencing of peptide library members and their corresponding tags has confirmed that the tags can accurately predict the peptide structure.

It is noted that peptide tags are more resistant to decomposition during ligand synthesis than are oligonucleotide tags, but they must be employed in molar ratios nearly equal to those of the ligand on typical 130 mm beads in order to be successfully sequenced. As with oligonucleotide encoding, the use of peptides as tags requires complex protection/deprotection chemistries.

2) Non-sequenceable Tagging: Binary Encoding

An alternative form of encoding the test peptide library employs a set of non-sequenceable electrophoric tagging molecules that are used as a binary code (Ohlmeyer et al. (1993) PNAS 90:10922-010926). Exemplary tags are haloaromatic alkyl ethers that are detectable as their tetramethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 240 (e.g., upwards of 1012) different molecules. In the original report (Ohlmeyer et al., supra) the tags were bound to about 1% of the available amine groups of a peptide library via a photocleavable O-nitrobenzyl linker. This approach is convenient when preparing combinatorial libraries of peptides or other amine-containing molecules. A more versatile system has, however, been developed that permits encoding of essentially any combinatorial library. Here, the ligand is attached to the solid support via the photocleavable linker and the tag is attached through a catechol ether linker via carbene insertion into the bead matrix (Nestler et al. (1994) J Org Chem 59:4723-4724). This orthogonal attachment strategy permits the selective detachment of library members for bioassay in solution and subsequent decoding by ECGC after oxidative detachment of the tag sets.

Binary encoding with electrophoric tags has been particularly useful in defining selective interactions of substrates with synthetic receptors (Borchardt et al. (1994) J Am Chem Soc 116:373-374), and model systems for understanding the binding and catalysis of biomolecules. Even using detailed molecular modeling, the identification of the selectivity preferences for synthetic receptors has required the manual synthesis of dozens of potential substrates. The use of encoded libraries makes it possible to rapidly examine all the members of a potential binding set. The use of binary-encoded libraries has made the determination of binding selectivities so facile that structural selectivity has been reported for four novel synthetic macrobicyclic and tricyclic receptors in a single communication (Wennemers et al. (1995) J Org Chem 60:1108-1109; and Yoon et al. (1994) Tetrahedron Lett 35:8557-8560) using the encoded library mentioned above. Similar facility in defining specificity of interaction would be expected for many other biomolecules.

Although the several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself Two binary-encoded combinatorial libraries have been reported where the electrophoric tags are attached directly to the solid phase (Ohlmeyer et al. (1995) PNAS 92:6027-6031) and provide guidance for generating the subject peptide library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, ligands are partially detached and transferred to assay plates; third, a bioassay identifies the active wells; fourth, the corresponding beads are rearrayed singly into new microtiter plates; fifth, single active compounds are identified; and sixth, the structures are decoded.

The above approach was employed in screening for carbonic anhydrase (CA) binding and identified compounds which exhibited nanomolar affinities for CA. Unlike sequenceable tagging, a large number of structures can be rapidly decoded from binary-encoded libraries (a single ECGC apparatus can decode 50 structures per day). Thus, binary-encoded libraries can be used for the rapid analysis of structure-activity relationships and optimization of both potency and selectivity of an active series. The synthesis and screening of large unbiased binary encoded peptide libraries for lead identification, followed by preparation and analysis of smaller focused libraries for lead optimization, offers a particularly powerful approach to drug discovery using the subject method.

iii) Nucleic Acid Libraries

In another embodiment, the library is comprised of a variegated pool of nucleic acids, e.g. single or double-stranded DNA or ARNA. A variety of techniques are known in the art for generating screenable nucleic acid libraries which may be exploited in the present invention. In particular, many of the techniques described above for synthetic peptide libraries can be used to generate nucleic acid libraries of a variety of formats. For example, divide-couple-recombine techniques can be used in conjugation with standard nucleic acid synthesis techniques to generate bead immobilized nucleic acid libraries.

In another embodiment, solution libraries of nucleic acids can be generated which rely on PCR techniques to amplify for sequencing those nucleic acid molecules which selectively bind the screening target. By such techniques, libraries approaching 1015 different nucleotide sequences have been generated in solution (see, for example, Bartel and Szostak (1993) Science 261:1411-1418; Bock et al. (1992) Nature 355:564; Ellington et al. (1992) Nature 355:850-852; and Oliphant et al. (1989) Mol Cell Biol 9:2944-2949).

According to one embodiment of the subject method, the SELEX (systematic evolution of ligands by exponential enrichment) is employed with the enantiomeric screening target. See, for example, Tuerk et al. (1990) Science 249:505-510 for a review of SELEX. Briefly, in the first step of these experiments on a pool of variant nucleic acid sequences is created, e.g. as a random or semi-random library. In general, an invariant 3′ and (optionally) 5′ primer sequence are provided for use with PCR anchors or for permitting subcloning. The nucleic acid library is applied to screening a target, and nucleic acids which selectively bind (or otherwise act on the target) are isolated from the pool. the isolates are amplified by PCR and subcloned into, for example, phagemids. The phagemids are then transfected into bacterial cells, and individual isolates can be obtained and the sequence of the nucleic acid cloned from the screening pool can be determined.

When RNA is the test ligand, the RNA library can be directly synthesized by standard organic chemistry, or can be provided by in vitro translation as described by Tuerk et al., supra. Likewise, RNA isolated by binding to the screening target can be reverse transcribed and the resulting cDNA subcloned and sequenced as above.

iv) Small Molecule Libraries

Recent trends in the search for novel pharmacological agents have focused on the preparation of chemical libraries. Peptide, nucleic acid, and saccharide libraries are described above. However, the field of combinatorial chemistry has also provided large numbers of non-polymeric, small organic molecule libraries which can be employed in the subject method.

Exemplary combinatorial libraries include benzodiazepines, peptoids, biaryls and hydantoins. In general, the same techniques described above for the various formats of chemically synthesized peptide libraries are also used to generate and (optionally) encode synthetic non-peptide libraries.

B. Selecting Compounds from the Library

As with the diversity contemplated for the screening target and form in which the compound library is provided, the subject method is envisaged with a variety of detection methods for isolating and identifying compounds which interact with the screening target. In most embodiments, the screening programs which test libraries of compounds will be derived for high throughput analysis in order to maximize the number of compounds surveyed in a given period of time. However, as a general rule, the screening portion of the subject method involves contacting the screening target with the compound library and isolating those compounds from the library which interact with the screening target. Such interaction may be detected, for example, based on directly detecting the binding of the compounds to the screening target, or inferred through the modulation of interactions involving the screening target with other molecules, such as protein-protein or protein-DNA interaction involving the screening target or modulation of an enzymatic/catalytic activity of the screening target. The efficacy of the test compounds can be assessed by generating dose response curves from data obtained using various concentrations of the test compound. Moreover, a control assay can also be performed to provide a baseline for comparison.

Complex formation between a test compounds and a screening target may be directly detected by a variety of techniques. The complexes can be scored for using, for example, detectably labeled compounds or screening targets, such as radiolabeled, fluorescently labeled, or enzymatically labeled polypeptides, by immunoassay, or by chromatographic detection.

In one embodiment, the variegated compound library is subjected to affinity enrichment in order to select for compounds which bind a preselected screening target. The term “affinity separation” or “affinity enrichment” includes, but is not limited to (1) affinity chromatography utilizing immobilizing screening targets, (2) precipitation using screening targets, (3) fluorescence activated cell sorting where the compound library is so amenable, (4) agglutination, and (5) plaque lifts. In each embodiment, the library of compounds are ultimately separated based on the ability of a particular compound to bind a screening target of interest. See, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; and the Ladner et al. International Publication No. WO 90/02809.

With respect to affinity chromatography, it will be generally understood by those skilled in the art that a great number of chromatography techniques can be adapted for use in the present invention, ranging from column chromatography to batch elution, and including ELISA and reverse biopanning techniques. Typically the screening target is immobilized on an insoluble carrier, such as sepharose or polyacrylamide beads, or, alternatively, the wells of a microtitre plate.

The population of compounds is applied to the affinity matrix under conditions compatible with the binding of compounds in the library to the immobilized screening target. The population is then fractionated by washing with a solute that does not greatly effect specific binding of compounds to the screening target, but which substantially disrupts any non-specific binding of components the library to the screening target or matrix. A certain degree of control can be exerted over the binding characteristics of the compounds recovered from the library by adjusting the conditions of the binding incubation and subsequent washing. The temperature, pH ionic strength, divalent cation concentration, and the volume and duration of the washing can select for compounds within a particular range of affinity and specificity. Selection based on slow dissociation rate, which is usually predictive of high affinity, is a very practical route. This may be done either by continued incubation in the presence of a saturating amount of free screening target, or by increasing the volume, number, and length of the washes. In each case, the rebinding of dissociated compounds from the applied library is prevented, and with increasing time, compounds of higher and higher affinity are recovered. Moreover, additional modifications of the binding and washing procedures may be applied to find compounds with special characteristics. The affinities of some compounds may be dependent on ionic strength or cation concentration. Specific examples are peptides which depend on Ca²⁺ or other ions for binding activity and which release from the screening target in the presence of a chelating agent such as EGTA. (see, Hopp et al. (1988) Biotechnology 6:1204-1210). Such peptides may be identified in the compound library by a double screening technique isolating first those that bind the screening target in the presence of Ca²⁺, and by subsequently identifying those in this group that fail to bind in the presence of EGTA.

After “washing” to remove non-specifically members of the compound library, when desired, specifically compounds can be eluted by either specific desorption (using excess screening target) or non-specific desorption (using pH, polarity reducing agents, or chaotropic agents). In preferred embodiments using biological display packages, the elution protocol does not kill the organism used as the display package such that the enriched population of display packages can be further amplified by reproduction. The list of potential eluants includes salts (such as those in which one of the counter ions is Na⁺, NH₄⁺, Rb⁺, So₄²⁻, H₂PO₄⁺, citrate, K⁺, Li⁺, Cs⁺, HSO₄⁻, CO₃²⁻, Ca²⁺, Sr²⁺, CL⁻, PO₄²⁻, HCO₃⁻, Mg²⁺, Ba²⁺, Br⁻, HPO₄²⁻, or acetate), acid, heat, and, when available, soluble forms of the target antigen (or analogs thereof). Because bacteria continue to metabolize during the affinity separation step and are generally more susceptible to damage by harsh conditions, the choice of buffer components (especially eluates) can be more restricted when the display package is a bacteria rather than for phage or spores. Neutral solutes, such as ethanol, acetone, ether, or urea, are examples of other agents useful for eluting the bound display packages.

In preferred embodiments of biological peptide displays or certain nucleic acid libraries, affinity enriched packages or nucleic acids are iteratively amplified and subjected to further rounds of affinity separation until enrichment of the desired binding activity is detected. In certain embodiments, the specifically bound biological display packages, especially bacterial cells, need not be eluted per se, but rather, the matrix bound display packages can be used directly to inoculate a suitable growth media for amplification.

Where the display package is a phage particle, the fusion protein generated with the coat protein can interfere substantially with the subsequent amplification of eluted phage particles, particularly in embodiments wherein the cpIII protein is used as the display anchor. Even though present in only one of the 5-6 tail fibers, some peptide constructs because of their size and/or sequence, may cause severe defects in the infectivity of their carrier phage. This causes a loss of phage from the population during reinfection and amplification following each cycle of panning. In one embodiment, the peptide can be derived on the surface of the display package so as to be susceptible to proteolytic cleavage which severs the covalent linkage of at least the antigen binding sites of the displayed peptide from the remaining package. For instance, where the cpIII coat protein of M13 is employed, such a strategy can be used to obtain infectious phage by treatment with an enzyme which cleaves between the peptide portion and cpIII portion of a tail fiber fusion protein (e.g. such as the use of an enterokinase cleavage recognition sequence).

To further minimize problems associated with defective infectivity, DNA prepared from the eluted phage can be transformed into host cells by electroporation or well known chemical means. The cells are cultivated for a period of time sufficient for marker expression, and selection is applied as typically done for DNA transformation. The colonies are amplified, and phage harvested for a subsequent round(s) of panning.

After isolation of biological display packages which encode peptides having a desired binding specificity for the screening target, the nucleic acid encoding the peptide for each of the purified display packages can be recloned in a suitable eukaryotic or prokaryotic expression vector and transfected into an appropriate host for production of large amounts of protein.

On the other hand, where chemically synthesized libraries are used in the form of display packages, the isolated peptides are identified either directly from the display, e.g., by direct microsequencing, or the display packages are appropriately decoded, e.g., by elucidating the identity of an associated tag/index. Deconvolution techniques are also known in the art.

It will be apparent that, in addition to utilizing binding as the separation criteria, compound libraries can be fractionated based on other activities of the target molecule, such as modulation of catalytic activity.

The practice of the present invention will employ, unless otherwise indicated, conventional techniques of cell biology, cell culture, molecular biology, microbiology and recombinant DNA, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987).

11. EXAMPLES

### Example 1

### Differential Display of Membrane Polypeptides

The present invention is further illustrated by the following examples which should not be construed as limiting in any way. The contents of all cited references (including literature references, issued patents, published patent applications as cited throughout this application} are hereby expressly incorporated by reference.

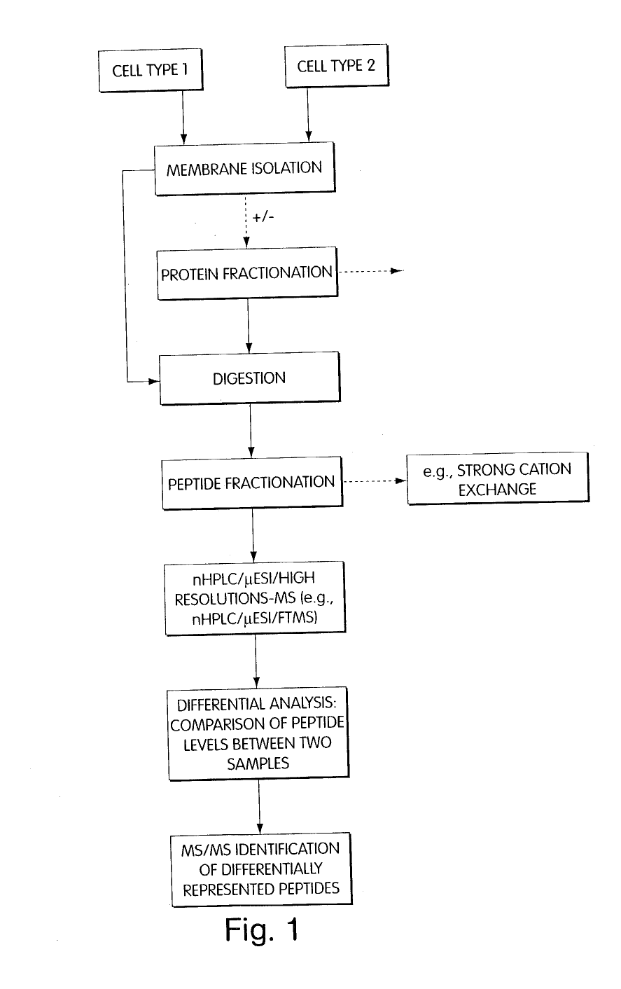

A gel-free proteomics approach therefore was used to examine cell surface polypeptides from breast cancer cell lines MCF-7 and SKBR3. The approach is carried out essentially as follows (see FIG. 1): Plasma membranes from each cell line were purified, and the polypeptides extracted and digested. The resulting peptides were then fractionated by strong cation exchange chromatography prior to differential analysis by nanoHPLC/microelectrospray ionization/Fourier transform mass spectrometry (nanoHPLC/μESI/FTMS). The first step in this differential analysis is the generation of a list of the peptides observed in the analysis of each cell line. The generation of this list requires the high resolution and large dynamic range intrinsic to FTMS data and also takes into account retention time. These lists for different samples (cell lines or different treatments) are then compared. Peptides that are observed at significantly higher levels in one sample are then subjected to targeted MS/MS with an LCQ ion trap mass spectrometer to determine their sequences and thus the identities of the parent polypeptides. This approach allowed the identification of a transmembrane tyrosine kinase receptor differentially expressed in the two cell types tested.

Materials and Methods

MCF-7 and SKBR3 (1×10⁸ cells each) were harvested and subjected to Dounce homogenization. Nuclei and cellular debris were removed by centrifugation. The plasma membrane fraction was then enriched by centrifugation at 25,000 rpm for 30 minutes in an SW41 rotor. Plasma membranes were resuspended in buffer containing protease inhibitors. Polypeptides were extracted from an aliquot of the plasma membrane fraction corresponding to 3×10⁷ cells by methanol/chloroform extraction. This aliquot was mixed vigorously with 3 volumes of methanol, 1 volume of chloroform, and 2 volumes of water; the suspension was then centrifuged and the resulting top layer discarded. The lower layer was mixed with three volumes of methanol and centrifuged. All liquid was removed and the resulting polypeptide pellet was dissolved in 0.1 M ammonium bicarbonate, pH 8, containing 0.1% SDS. Trypsin (20 μg) was added before incubation at 37° C. overnight.

Ion exchange was performed on the polypeptide digest to reduce the complexity of the mixture prior to MS analysis. The sample was first desalted by loading onto a desalting column (14 cm Poros R2 20 beads in 360 μm×200 μm fused silica) and rinsing with ca. 15 column volumes of 0.1% acetic acid. The peptides were then eluted with ca. 15 column volumes of 80% acetonitrile in 0.1% acetic acid. The sample was then concentrated to 5-10 μL and diluted to 100 μL with 0.1% acetic acid. To perform the ion exchange, the sample was loaded onto the ion exchange column (2 cm Poros HS 20 SCX media in 360 μm×200 μm fused silica) and rinsed with 100 μL 0.1% acetic acid. The sample was then step-eluted with 100 μL each 0, 2, 5, 10, 15, 25, 50, 75, 100, and 500 mM KCl in 5 mM K₂HPO₄/5% acetonitrile.

The 2 mM KCl fraction was subjected to differential MS analysis, 5% of this fraction was diluted with 2 volumes of 15% acetic acid, loaded to a reverse phase precolumn (5 cm 5-20 μm C18 beads in 360 μm×100 μm fused silica), and washed for 20 minutes with 0.1% acetic acid. The precolumn was then butt-connected to a reverse phase analytical column with a laser-pulled μESI emitter tip (1). Peptides were gradient-eluted (0-36% acetonitrile in 0.1% acetic acid in 40 minutes) into a homebuilt Fourier transform mass spectrometer. During this elution, 1250 high resolution mass spectra were collected.

To compare the peptides observed during these analyses, the spectra from the analysis of one sample are deconvoluted to generate a list of peptides. The other sample is then examined to determine the presence and level of those peptides, taking into account not only the mass of the peptide (required to be within 0.02 amu) but also the elution time. Peptides that appear to be present at >5-fold greater abundance in one sample are manually verified and then subjected to targeted collision-activated dissociation (CAD) on a quadrupole ion trap mass spectrometer (2). These spectra are then searched against polypeptide databases or manually interpreted to determine the sequence of the peptide and thus the identity of the differentially expressed parent polypeptide.

Results

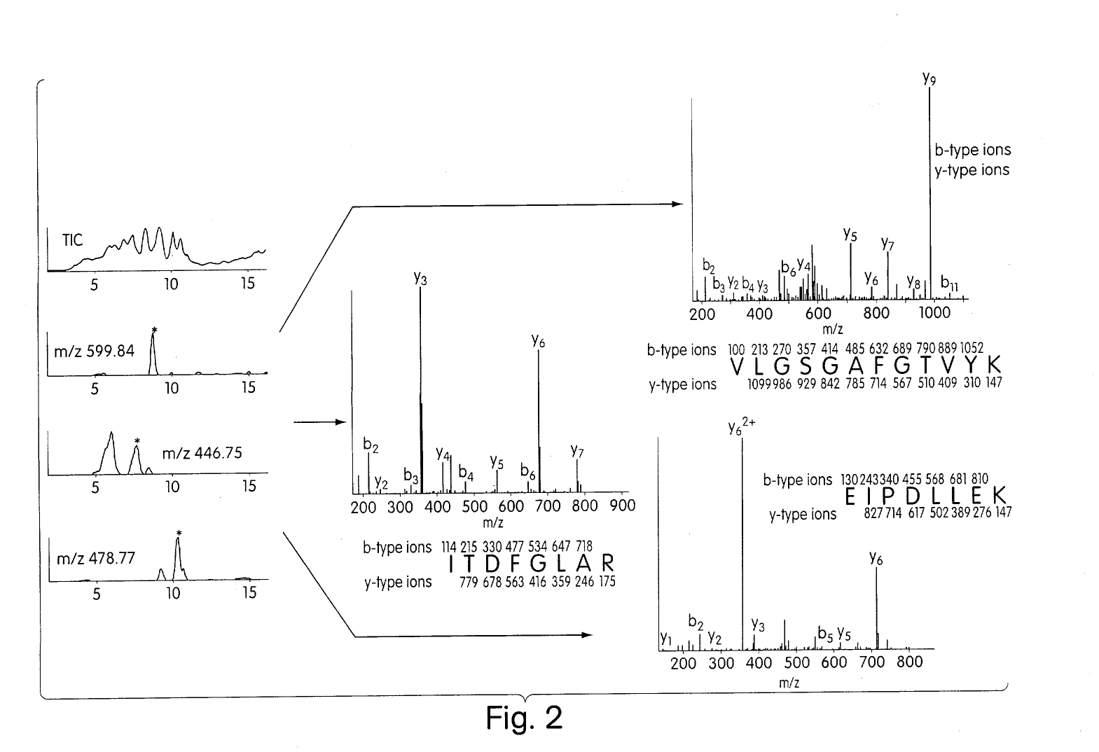

Comparison of the 2 mM KCl ion exchange fractions from MCF-7 and SKBR3 membrane preparations revealed the differential representation of >100 peptides. Three peptides observed at significantly higher levels (>10-fold) in an SKBR3 preparation had masses corresponding to tryptic peptides from the intracellular domain of the 185 kDa transmembrane tyrosine kinase Her2/neu, a product of the protooncogene ErbB2 that is known to be overexpressed in the SKBR3 cell line (3). CAD spectra of these peptides were obtained, identifying the peptides as VLGSGAFGTVYK (SEQ ID NO: 1) 725-736, ITDFGLAR (SEQ ID NO: 2) 861-868, and EIPDLLEK (SEQ ID NO: 3) 930-937 (see FIG. 2). These data reveal that differential MS analysis, relying on production of high resolution mass spectra, can be successfully applied to membrane polypeptides, including large glycosylated polypeptides with transmembrane domains like Her2/neu. Furthermore, the identification of multiple peptides from a single polypeptide indicates that the redundancy integral to this approach will help validate observations of polypeptide overexpression. Thus this methodology holds great potential for the identification of disease- or tissue-specific cell surface markers and potential drug targets.

### Example 2

### Differential Display of Phosphopeptides

Data that demonstrate new or increased polypeptide phosphorylation are a powerful complement to information about differential polypeptide expression, providing valuable indications of pathways activated upon cell transformation. The differential analysis approach described above can be applied to phosphopeptides from enzymatically digested membrane polypeptides. MCF-7 cells were treated with heregulin-alpha to activate ErbB receptors, including Her2/neu, and thus to induce phosphorylation cascades. Plasma membranes from treated and untreated cells were purified, the polypeptides digested, and resulting phosphopeptides isolated by immobilized metal affinity chromatography (IMAC) prior to differential analysis as described above. From approximately 7 minutes of analysis, 22 phosphopeptide species were observed to be present at >5-fold higher abundance in heregulin-treated cells than in untreated cells; the identification of phosphopeptides are performed by MS/MS.

These methodologies hold great potential both for the identification of disease-specific cell surface markers and for the determination of pathways activated during transformation.

### EQUIVALENTS

Those skilled in the art will recognize, or be able to ascertain using no more than routine experimentation, numerous equivalents to the specific procedures described herein. Such equivalents are considered to be within the scope of this invention and are covered by the following claims.

1. A method for identifying changes in membrane-associated polypeptides, comprising:
(i) providing a test sample of membrane-associated polypeptides isolated from a test cell(s);
(ii) by mass spectrometry using a quantitative mass analyzer, determining the levels of polypeptides in said test sample;
(iii) comparing the level of one or more of the polypeptides from said test sample with levels of respective polypeptides from a reference sample;
(iv) identifying the sequences of polypeptides in the test sample which, relative to the reference sample, have altered abundance and/or altered levels of post-translational modification.

2. The method of claim 1, wherein the levels of polypeptides in said test sample is determined by Fourier-transform ion cyclotron resonance mass spectrometry (FTMS).

3. The method of claim 1, wherein the levels of polypeptides in said test sample is determined by Time-of-Flight mass spectrometry (TOF-MS).

4. The method of claim 1, wherein the membrane-associated polypeptides are cleaved to produce fragments including C-terminal arginine or lysine residues prior to analysis by mass spectrometry.

5. The method of claim 1, wherein the membrane-associated polypeptides are separated by chromatography prior to analysis by mass spectrometry.

6. The method of claim 5, wherein the chromatography is strong cation exchange (SCX) chromatography.

7. The method of claim 1, wherein the mass spectrometry step includes ionizing the polypeptides of the test sample by electrospray ionization.

8. The method of claim 1, wherein the test sample is from a disease tissue and the reference sample is from a normal tissue.

9. The method of claim 1, wherein the polypeptides of the test sample are isolated based on post-translational modification.

10. The method of claim 9, wherein the polypeptides of the test sample are isolated based on phosphorylation.

11. A method for identification of membrane-associated polypeptide targets of a compound, comprising:
(i) providing two test samples of membrane-associated polypeptides isolated from two test cells, wherein one test sample is a reference sample and the other is a sample treated by said compound;
(ii) by mass spectrometry using a quantitative mass analyzer, determining the levels of polypeptides in said test samples;
(iii) comparing the level of one or more of the polypeptides from said treated test sample with levels of respective polypeptides from said reference sample;
(iv) identifying the sequences of polypeptides in said treated sample which, relative to the reference sample, have altered abundance and/or altered levels of post-translational modification(s), thereby identifying the membrane-associated polypeptide targets of said compound.

12. A method for identifying a compound which alters the abundance of a membrane-associated polypeptide in a sample, comprising:
(i) providing a reference sample and a plurality of test samples of membrane-associated polypeptides, each isolated from a test cell treated by a specific test compound;
(ii) by mass spectrometry using a quantitative mass analyzer, determining the levels of said membrane-associated polypeptides in said test samples and said reference samples;
(iii) comparing the level of one or more of said membrane-associated polypeptides from said test samples with levels of respective polypeptides from said reference sample;
(iv) identifying the test sample which, relative to the reference sample, have altered abundance, thereby identifying the test compound responsible for the change.

13. A method for identifying a compound which alters the levels of post-translational modification of a membrane-associated polypeptide in a sample, comprising:
(i) providing a reference sample and a plurality of test samples of membrane-associated polypeptides, each isolated from a test cell treated by a specific test compound;
(ii) by mass spectrometry using a quantitative mass analyzer, determining the levels of said membrane-associated polypeptides in said test samples and said reference samples;
(iii) comparing the level of one or more of said membrane-associated polypeptides from said test samples with levels of respective polypeptides from said reference sample;
(iv) identifying the test sample which, relative to the reference sample, have altered levels of post-translational modification, thereby identifying the test compound responsible for the change.

14. A method of conducting a pharmaceutical business, comprising:
(i) by the above-described method, determining the identity of a target polypeptide isolated on the basis of the polypeptide being (a) having a differential cellular localization of interest; (b) having a differential expression pattern of interest; (c) having a differential post-translational modification of interest; or (d) having a differential abundance of interest;
(ii) identifying compounds by their ability to alter the abundance or subcellular localization or post-translational modification of the target polypeptide;
(iii) conducting therapeutic profiling of compounds identified in step (ii), or further analogs thereof, for efficacy and toxicity in animals; and,
(iv) formulating a pharmaceutical preparation including one or more compounds identified in step (iii) as having an acceptable therapeutic profile.

15. The business method of claim 14, further comprising an additional step of establishing a distribution system for distributing the pharmaceutical preparation for sale.

16. The business method of claim 14, further including establishing a sales group for marketing the pharmaceutical preparation.

17. A method of conducting a pharmaceutical business, comprising:
(i) by the above-described method, determining the identity of a target polypeptide isolated on the basis of the polypeptide: (a) having a differential cellular localization of interest, (b) having a differential expression pattern of interest, (c) having a differential post-translational modification of interest, or (d) having a differential abundance of interest;
(ii) (optionally) conducting therapeutic profiling of the target gene for efficacy and toxicity in animals; and
(iii) licensing, to a third party, the rights for further drug development of inhibitors or activators of the target gene.
